(19) United States
(12) Patent Application Publication
(10) Pub. No.: US 2023/0019986 A1
(43) Pub. Date: Jan. 19, 2023

(54) COMPOSITIONS AND METHODS FOR REDUCING CYTOKINE EXPRESSION

(71) Applicant: Evelo Biosciences, Inc., Cambridge, MA (US)

(72) Inventors: David Epstein, Cambridge, MA (US); Duncan McHale, Kent (GB)

(21) Appl. No.: 17/905,001

(22) PCT Filed: Feb. 26, 2021
(86) PCT No.: PCT/US21/19968

§ 371 Date: Aug. 25, 2022

Related U.S. Application Data
(60) Provisional application No. 63/074,429, filed on Sep. 3, 2020.
(60) Provisional application No. 63/053,916, filed on Jul. 20, 2020.
(60) Provisional application No. 63/021,224, filed on May 7, 2020.
(60) Provisional application No. 63/000,201, filed on Mar. 26, 2020.
(60) Provisional application No. 62/983,091, filed on Feb. 28, 2020.
(60) Provisional application No. 62/981,867, filed on Feb. 26, 2020.

(51) Int. Cl. A61K 35/741; A61K 9/00; A61K 31/4706; A61K 31/7056; A61K 38/21; A61K 31/215; A61K 31/675; A61K 39/395; A61K 31/351; A61K 31/196; A61K 31/53; A61K 45/06; A61P 11/00; A61P 31/14; A61P 37/06; A61K 9/20; A61K 9/28; A61K 9/48

(52) U.S. Cl. CPC A61K 35/741; A61K 9/0053; A61K 31/4706; A61K 31/7056; A61K 38/21; A61K 31/215; A61K 31/675; A61K 39/3955; A61K 31/351; A61K 31/196; A61K 31/53; A61K 45/06; A61P 11/00; A61P 31/14; A61P 37/06; A61K 9/2009; A61K 9/2013; A61K 9/2018; A61K 9/2054; A61K 9/2027; A61K 9/2813; A61K 9/2846; A61K 9/4858; A61K 9/485

(57) ABSTRACT

Provided herein are methods and compositions related to Prevotella bacteria for the reduction of IL-8, IL-6, IL-Iβ, and/C or TNFα expression and/or for the treatment of viral infections.

### RELATED APPLICATIONS

This application claims the benefit of priority to U.S. Provisional Patent Applications having Ser. Nos. 63/074,429 filed Sep. 3, 2020, 63/053,916 filed Jul. 20, 2020, 63/021,224 filed May 7, 2020, 63/000,201 filed Mar. 26, 2020, 62/983,091 filed Feb. 28, 2020, and 62/981,867 filed Feb. 26, 2020, the entire contents of each of which are hereby incorporated by reference in their entirety.

### BACKGROUND

Inflammation can be a protective response to harmful stimuli, such as invading pathogens, damaged cells, toxic compounds, or cancerous cells. However excessive inflammatory responses to such stimuli can result in serious adverse effects, including tissue damage and even death. For example, production of pro-inflammatory cytokines such as interleukin-8 (IL-8), interleukin-6 (IL-6), interleukin-1 beta (IL-1β), and tumor necrosis factor alpha (TNFα) in response to many viral infections is one of the primary causes of the adverse symptoms associated with infection (including, in some cases, death). For example, release of inflammatory cytokines has been associated with disease severity resulting from infection by a number of viruses, including infection by coronaviruses (e.g., SARS-CoV-2, the virus that causes Coronavirus Disease 2019 (COVID-19)), influenza viruses, and respiratory syncytial viruses. For example, patients with severe COVID-19 often exhibit elevated levels of inflammatory cytokines in their lungs, which contributes to lung damage experienced by the COVID-19 patients.

Thus, there is a great need for new compositions and methods for the reduction of inflammatory cytokine expression, particularly in subjects who have been infected by a respiratory virus and/or who have an increased risk of being infected by a respiratory virus.

### SUMMARY

Provided herein are methods and compositions related to the use of certain strains of Prevotella histicola for the reduction of inflammatory cytokine expression (e.g., IL-8, IL-6, IL-1β, and/or TNFα expression) and/or for the treatment of bacterial septic shock, cytokine storm and/or viral infection. In some embodiments, the methods and compositions provided herein are for the reduction of inflammatory cytokine expression (e.g., IL-8, IL-6, IL-1β, and/or TNFα expression) and/or for the treatment of a viral infection such as a respiratory viral infection, such as a coronavirus infection (e.g., a MERS (Middle East Respiratory Syndrome) infection, a severe acute respiratory syndrome (SARS) infection, such as a SARS-CoV-2 infection), an influenza infection, and/or a respiratory syncytial virus infection. In some embodiments, the methods and compositions provided herein are for the treatment of a coronavirus infection (e.g., a MERS infection, a severe acute respiratory syndrome (SARS) infection, such as a SARS-CoV-2 infection). In some embodiments, provided herein are methods of treating COVID-19. In some embodiments, the methods and compositions provided herein are for the treatment of an influenza virus infection.

In certain aspects, provided herein is a method of reducing inflammatory cytokine expression (e.g., reducing IL-8, IL-6, IL-1β, and/or TNFα expression levels) in a subject in need thereof, comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329). In certain aspects, provided herein is a method of reducing IL-8 expression levels. In certain aspects, provided herein is a method of reducing IL-6 expression levels. In certain aspects, provided herein is a method of reducing IL-1β expression levels. In certain aspects, provided herein is a method of reducing TNFα expression levels. In certain aspects, provided herein is a method of reducing IL-8 and IL-6 expression levels. In certain aspects, provided herein is a method of reducing IL-8, IL-6, and TNFα expression levels.

In certain aspects, provided herein is a method of reducing inflammatory cytokine expression (e.g., reducing IL-8, IL-6, IL-1β, and/or TNFα expression levels) in a subject in need thereof, comprising administering to the subject a Prevotella histicola strain comprising at least 99% genomic, 16S and/or CRISPR sequence identity to the nucleotide sequence of the Prevotella histicola Strain B (NRRL accession number B 50329), wherein a Type 1 interferon response is not reduced (e.g., not reduced to the same extent that the inflammatory cytokine expression is reduced), e.g., as determined by IFNα and/or IFNβ levels. In some embodiments, the Prevotella histicola strain is administered in a pharmaceutical composition and/or a solid dosage form. In some embodiments, the Prevotella histicola strain is Prevotella histicola Strain B (NRRL accession number B 50329).

In certain aspects, provided herein is a method of treating a viral infection in a subject comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329). In some embodiments, the viral infection is a coronavirus infection, an influenza infection, and/or a respiratory syncytial virus infection. In some embodiments the viral infection is a SARS-CoV-2 infection.

In certain aspects, provided herein is a method of treating COVID-19 in a subject comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329).

In certain aspects, provided herein is a method of treating cytokine storm syndrome (cytokine release syndrome) (e.g., a cytokine storm resulting from a viral infection, such as a SARS-CoV-2 infection) in a subject comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329).

In some embodiments of the methods provided herein, the method improves pulmonary function in the subject, as measured by the change in Oxygen Saturation (SpO2)/Fraction of Inspired Oxygen (FiO2) [S/F] ratio, e.g., as measured by a change from baseline to the lowest S/F ratio measured in days 1-14 as described herein.

In some embodiments of the methods provided herein, the method improves a clinical endpoint in a subject, e.g., an endpoint described herein, e.g., an endpoint provided in Table 1.

In some embodiments of the methods provided herein, the method decreases development of complications of COVID-19 infection, e.g., as described herein.

In some embodiments of the methods provided herein, the method decreases severity of complications of COVID-19 infection, e.g., as described herein.

In some embodiments of the methods provided herein, the method improves the WHO OSCI score in a subject, e.g., evaluated as described herein.

In some embodiments of the methods provided herein, the method decreases length of hospitalization in subjects with COVID-19, e.g., as described herein.

In some embodiments of the methods provided herein, the method decreases length of recovery in subjects with COVID-19, e.g., as described herein.

In some embodiments of the methods provided herein, the method decreases the exaggerated host cytokine response to COVID-19 infection, e.g., as determined by change from baseline in a cytokine level (such as IL-8, IL-6, IL-1β, and/or TNFα) at day 4 and/or day 7 and/or by change from baseline in inflammatory response at day 4 and/or day 7, e.g., as described herein. In some embodiments, the method decreases the exaggerated host cytokine response to COVID-19 infection, e.g., as determined by change from baseline in IL-6 levels at day 4 and/or day 7, e.g., as described herein.

In some embodiments of the methods provided herein, the method causes a change in a biomarker, e.g., a biomarker described herein, e.g., as determined by change from baseline in the biomarker at day 4 and day 7. The biomarker can be, for example, one or more of: differential white cell count, neutrophil to lymphocyte ratio, CRP, IL-6, IL-8, Ferritin, D-Dimer, Troponin, Eotaxin, Eotaxin-3, GM-CSF, IFN-γ, IL-1α, IL-1β, IL-2, IL-4, IL-5, IL-7, IL-8 (HA), IL-10, IL-12/IL-23p40, IL-12p70, IL-13, IL-15, IL-16, IL-17A, IP-10, MCP-1, MCP-4, MDC, MIP-1α, MIP-1β, TARC, TNF-α, TNF-β, and/or VEGF-A levels (e.g., protein or mRNA levels).

In some embodiments, the pharmaceutical compositions comprise whole Prevotella histicola bacteria (e.g., live bacteria, killed bacteria, attenuated bacteria).

In certain embodiments of the methods provided herein, at least 4×10¹⁰ cells of the Prevotella histicola strain are administered to the subject daily. In some embodiments, at least 4×10¹⁰ cells, 5×10¹⁰ cells, 6×10¹⁰ cells, 7×10¹⁰ cells, 8×10¹⁰ cells, 9×10¹⁰ cells, 1.0×10¹¹ cells, 1.1×10¹¹ cells, 1.2×10¹¹ cells, 1.3×10¹¹ cells, 1.4×10¹¹ cells, 1.5×10¹¹ cells, 1.6×10¹¹ cells, 1.7×10¹¹ cells, 1.8×10¹¹ cells, 1.9×10¹¹ cells, 2.0×10¹¹ cells, 2.1×10¹¹ cells, 2.2×10¹¹ cells, 2.3×10¹¹ cells, 2.4×10¹¹ cells, 2.5×10¹¹ cells, 2.6×10¹¹ cells, 2.7×10¹¹ cells, 2.8×10¹¹ cells, 2.9×10¹¹ cells, 3.0×10¹¹ cells, 3.1×10¹¹ cells, 3.2×10¹¹ cells, 3.3×10¹¹ cells, 3.4×10¹¹ cells, 3.5×10¹¹ cells, 3.6×10¹¹ cells, 3.7×10¹¹ cells, 3.8×10¹¹ cells, 3.9×10¹¹ cells, 4.0×10¹¹ cells, 4.1×10¹¹ cells, 4.2×10¹¹ cells, 4.3×10¹¹ cells, 4.4×10¹¹ cells, 4.5×10¹¹ cells, 4.6×10¹¹ cells, 4.7×10¹¹ cells, 4.8×10¹¹ cells, 4.9×10¹¹ cells, 5.0×10¹¹ cells, 5.1×10¹¹ cells, 5.2×10¹¹ cells, 5.3×10¹¹ cells, 5.4×10¹¹ cells, 5.5×10¹¹ cells, 5.6×10¹¹ cells, 5.7×10¹¹ cells, 5.8×10¹¹ cells, 5.9×10¹¹ cells, 6.0×10¹¹ cells, 6.1×10¹¹ cells, 6.2×10¹¹ cells, 6.3×10¹¹ cells, 6.4×10¹¹ cells, 6.5×10¹¹ cells, 6.6×10¹¹ cells, 6.7×10¹¹ cells, 6.8×10¹¹ cells, 6.9×10¹¹ cells, 7.0×10¹¹ cells, 7.1×10¹¹ cells, 7.2×10¹¹ cells, 7.3×10¹¹ cells, 7.4×10¹¹ cells, 7.5×10¹¹ cells, 7.6×10¹¹ cells, 7.7×10¹¹ cells, 7.8×10¹¹ cells, 7.9×10¹¹ cells, or 8.0×10¹¹ cells of the Prevotella histicola strain are administered to the subject daily. In some embodiments, from 4×10¹⁰ cells to 1.6×10¹² cells of the Prevotella histicola strain are administered to the subject daily. In certain embodiments, from 4×10¹⁰ cells to 8×10¹¹ cells of the Prevotella histicola strain are administered to the subject daily. In certain embodiments, from 1.6×10¹⁰ cells to 16×10¹¹ cells of the Prevotella histicola strain are administered to the subject daily. In certain embodiments, from 8×10¹¹ cells to 16×10¹¹ cells of the Prevotella histicola strain are administered to the subject daily. In certain embodiments, from 8×10¹⁰ cells to 8×10¹¹ cells of the Prevotella histicola strain are administered to the subject daily. In certain embodiments, from 8×10¹⁰ cells to 1.6×10¹¹ cells of the Prevotella histicola strain are administered to the subject daily. In certain embodiments, from 1.6×10¹¹ cells to 8×10¹¹ cells of the Prevotella histicola strain are administered to the subject daily. In some embodiments, about 8×10¹⁰ cells of the Prevotella histicola strain are administered to the subject daily. In some embodiments, about 1.6×10¹¹ cells of the Prevotella histicola strain are administered to the subject daily. In some embodiments, about 3.2×10¹¹ cells of the Prevotella histicola strain are administered to the subject daily. In some embodiments, about 8×10¹¹ cells of the Prevotella histicola strain are administered to the subject daily. In some embodiments, about 1.6×10¹¹ cells of the Prevotella histicola strain are administered to the subject once daily. In some embodiments, about 1.6×10¹¹ cells of the Prevotella histicola strain are administered to the subject twice daily. In some embodiments, about 1.6×10¹¹ cells of the Prevotella histicola strain are administered to the subject twice daily (e.g., for 1-7 days, 3 days, 7 days, 10 days, or 14 days), and then about 1.6×10¹¹ cells of the Prevotella histicola strain are administered to the subject once daily, e.g., for the duration of the treatment period (e.g., up to 14 days of total treatment).

In some embodiments, about 9.6×10¹¹total cells of the Prevotella histicola strain are administered to the subject daily.

In some embodiments, about 12.8×10¹¹total cells of the Prevotella histicola strain are administered to the subject daily.

In some embodiments, about 16×10¹¹total cells of the Prevotella histicola strain are administered to the subject daily.

In some embodiments, about 9.6×10¹¹to about 16×10¹¹ total cells of the Prevotella histicola strain are administered to the subject daily.

In some embodiments, about 9.6×10¹¹to about 12.8×10¹¹ total cells of the Prevotella histicola strain are administered to the subject daily.

In some embodiments, about 12.8×10¹¹to about 16×10¹¹ total cells of the Prevotella histicola strain are administered to the subject daily.

In some embodiments, total cells of the Prevotella histicola strain are administered as a pharmaceutical composition.

In some embodiments, the pharmaceutical composition comprises one strain of bacteria, wherein the one strain of bacteria is a strain comprising at least 99% sequence identity to the nucleotide sequence of the Prevotella histicola Strain B 50329 (NRRL accession number B 50329). In some embodiments, the pharmaceutical composition comprises one strain of bacteria, wherein the one strain of bacteria is the Prevotella histicola Strain B 50329 (NRRL accession number B 50329).

In some embodiments, the pharmaceutical composition comprises about 1.6×10¹⁰ total cells of Prevotella histicola , e.g., of Prevotella Strain B 50329.

In some embodiments, the pharmaceutical composition comprises about 8×10¹⁰ total cells of Prevotella histicola , e.g., of Prevotella Strain B 50329.

In some embodiments, the pharmaceutical composition comprises about 1.6×10¹¹ total cells of Prevotella histicola , e.g., of Prevotella Strain B 50329.

In some embodiments, the pharmaceutical composition comprises about 3.2×10¹¹ total cells of Prevotella histicola , e.g., of Prevotella Strain B 50329.

In some embodiments, the pharmaceutical composition comprises about 8×10¹¹ total cells of Prevotella histicola , e.g., of Prevotella Strain B 50329.

In some embodiments, the pharmaceutical composition comprises about 1.6×10¹⁰ to about 8×10¹¹ total cells of Prevotella histicola , e.g., of Prevotella Strain B 50329.

In some embodiments, the pharmaceutical composition comprises about 1.6×10¹⁰ to about 16×10¹¹ total cells of Prevotella histicola , e.g., of Prevotella Strain B 50329.

In some embodiments, the pharmaceutical composition comprises about 1.6×10¹⁰ to about 1.6×10¹¹ total cells of Prevotella histicola , e.g., of Prevotella Strain B 50329.

In some embodiments, the pharmaceutical composition comprises about 1.6×10¹¹ to about 8×10¹¹ total cells of Prevotella histicola , e.g., of Prevotella Strain B 50329.

In some embodiments, the pharmaceutical composition comprises about 8×10¹⁰ to about 8×10¹¹ total cells of Prevotella histicola , e.g., of Prevotella Strain B 50329.

In certain embodiments, the pharmaceutical composition (e.g., composition of the total dose administered, e.g., once or twice daily) comprises at least 1×10¹⁰ total cells (e.g., at least 1×10¹⁰ total cells, at least 2×10¹⁰ total cells, at least 3×10¹⁰ total cells, at least 4×10¹⁰ total cells, at least 5×10¹⁰ total cells, at least 6×10¹⁰ total cells, at least 7×10¹⁰ total cells, at least 8×10¹⁰ total cells, at least 9×10¹⁰ total cells, at least 1×10¹¹ total cells of the Prevotella bacteria. In some embodiments, the pharmaceutical composition comprises no more than 9×10¹¹ total cells (e.g., no more than 1×10¹⁰ total cells, no more than 2×10¹⁰ total cells, no more than 3×10¹⁰ total cells, no more than 4×10¹⁰ total cells, no more than 5×10¹⁰ total cells, no more than 6×10¹⁰ total cells, no more than 7×10¹⁰ total cells, no more than 8×10¹⁰ total cells, no more than 9×10¹⁰ total cells, no more than 1×10¹¹ total cells, no more than 2×10¹¹ total cells, no more than 3×10¹¹ total cells, no more than 4×10¹¹ total cells, no more than 5×10¹¹ total cells, no more than 6×10¹¹ total cells, no more than 7×10¹¹ total cells, no more than 8×10¹¹ total cells) of the Prevotella bacteria. In some embodiments, the pharmaceutical composition comprises about 6×10⁹ total cells of the Prevotella bacteria.

In some embodiments, the pharmaceutical composition comprises about 1.6×10¹⁰ total cells of the Prevotella bacteria. In some embodiments, the pharmaceutical composition comprises about 8×10¹⁰ total cells of the Prevotella bacteria. In some embodiments, the pharmaceutical composition comprises about 1.6×10¹¹ total cells the Prevotella bacteria. In some embodiments, the pharmaceutical composition comprises about 3.2×10¹¹ total cells the Prevotella bacteria. In some embodiments, the pharmaceutical composition comprises about 8×10¹¹ total cells of the Prevotella bacteria. In some embodiments, the pharmaceutical composition comprises about 1.6×10¹⁰ to about 8×10¹¹ total cells of the Prevotella bacteria. In some embodiments, the pharmaceutical composition comprises about 1.6×10¹⁰ to about 1.6×10¹¹ total cells of the Prevotella bacteria. In some embodiments, the pharmaceutical composition comprises about 1.6×10¹⁰ to about 16×10¹¹ total cells of the Prevotella bacteria. In some embodiments, the pharmaceutical composition comprises about 8×10¹⁰ to about 8×10¹¹ total cells of the Prevotella bacteria. In some embodiments, the pharmaceutical composition comprises about 1.6×10¹¹ to about 8×10¹¹ total cells of the Prevotella bacteria.

In some embodiments, the pharmaceutical composition comprises about 9.6×10¹¹ total cells of the Prevotella bacteria.

In some embodiments, the pharmaceutical composition comprises about 12.8×10¹¹ total cells of the Prevotella bacteria.

In some embodiments, the pharmaceutical composition comprises about 16×10¹¹ total cells of the Prevotella bacteria.

In some embodiments, the pharmaceutical composition comprises about 9.6×10¹¹ to about 16×10¹¹ total cells of the Prevotella bacteria.

In some embodiments, the pharmaceutical composition comprises about 9.6×10¹¹ to about 12.8×10¹¹ total cells of the Prevotella bacteria.

In some embodiments, the pharmaceutical composition comprises about 12.8×10¹¹ to about 16×10¹¹ total cells of the Prevotella bacteria.

In certain embodiments, the pharmaceutical composition is provided as a solid dosage form (also referred to as a solid dose form). In some embodiments, provided herein are solid dosage forms comprising the Prevotella bacteria. In some embodiments, the solid dosage form comprises an enteric coating (e.g., HPMC coat).

In some embodiments, the solid dosage form comprises a capsule. In some embodiments, the capsule is an enteric coated capsule. In some embodiments, the enteric coating comprises HPMC. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

In some embodiments, 1, 2, 3, 4, 5, 6, 7, 8, 9, or 10 capsules are administered, e.g., once or twice daily to a subject.

In some embodiments, the Prevotella bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, each capsule comprises about 1.6×10¹⁰ total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 1.6×10¹⁰ total cells) is administered, e.g., once or twice daily to a subject. In some embodiments, 2 capsules (e.g., each comprising about 1.6×10¹⁰ total cells) are administered, e.g., once or twice daily to a subject. In some embodiments, 4 capsules (e.g., each comprising about 1.6×10¹⁰ total cells) are administered, e.g., once or twice daily to a subject. In some embodiments, 5 capsules (e.g., each comprising about 1.6×10¹⁰ total cells) are administered, e.g., once or twice daily to a subject. In some embodiments, 10 capsules (e.g., each comprising about 1.6×10¹⁰ total cells) are administered, e.g., once or twice daily to a subject.

In some embodiments, each capsule comprises about 8×10¹⁰ total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 8×10¹⁰ total cells) is administered, e.g., once or twice daily to a subject. In some embodiments, 2 capsules (e.g., each comprising about 8×10¹⁰ total cells) are administered, e.g., once or twice daily to a subject. In some embodiments, 4 capsules (e.g., each comprising about 8×10¹⁰ total cells) are administered, e.g., once or twice daily to a subject. . In some embodiments, 5 capsules (e.g., each comprising about 8×10¹⁰ total cells) are administered, e.g., once or twice daily to a subject. In some embodiments, 10 capsules (e.g., each comprising about 8×10¹⁰ total cells) are administered, e.g., once or twice daily to a subject.

In some embodiments, each capsule comprises about 1.6×10¹¹total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 1.6×10¹¹total cells) is administered, e.g., once or twice daily to a subject. In some embodiments, 2 capsules (e.g., each comprising about 1.6×10¹¹ total cells) are administered, e.g., once or twice daily to a subject. In some embodiments, 4 capsules (e.g., each comprising about 1.6×10¹¹total cells) are administered, e.g., once or twice daily to a subject. In some embodiments, 5 capsules (e.g., each comprising about 1.6×10¹¹total cells) are administered, e.g., once or twice daily to a subject. In some embodiments, 10 capsules (e.g., each comprising about 1.6×10¹¹ total cells) are administered, e.g., once or twice daily to a subject.

In some embodiments, each capsule comprises about 3.2×10¹¹total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 3.2×10¹¹total cells) is administered, e.g., once or twice daily to a subject. In some embodiments, 2 capsules (e.g., each comprising about 3.2×10¹¹ total cells) are administered, e.g., once or twice daily to a subject. In some embodiments, 5 capsules (e.g., each comprising about 3.2×10¹¹total cells) are administered, e.g., once or twice daily to a subject. In some embodiments, 10 capsules (e.g., each comprising about 3.2×10¹¹total cells) are administered, e.g., once or twice daily to a subject.

In some embodiments, the solid dosage form comprises a tablet. In some embodiments, the tablet is an enteric coated tablet. In some embodiments, the tablet is from 5 mm to 18 mm in diameter. In some embodiments, the enteric coating comprises HPMC. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

In some embodiments, 1, 2, 3, 4, 5, 6, 7, 8, 9, or 10 tablets are administered, e.g., once or twice daily to a subject.

In some embodiments, the Prevotella bacteria in the tablet are lyophilized. In some embodiments, the Prevotella bacteria in the tablet are lyophilized (e.g., in a powder). In some embodiments, the Prevotella bacteria in the tablet are lyophilized in a powder, and the powder further comprises mannitol, magnesium stearate, and/or colloidal silicon dioxide.

In some embodiments, the tablet comprises about 8×10¹⁰ total cells of the Prevotella bacteria (e.g., total dose of a tablet or plurality of tablets).

In some embodiments, the tablet comprises about 1.6×10¹¹ total cells of the Prevotella bacteria (e.g., total dose of a tablet or plurality of tablets).

In some embodiments, the tablet comprises about 3.2×10¹¹ total cells of the Prevotella bacteria (e.g., total dose of a tablet or plurality of tablets).

In some embodiments, the tablet comprises about 8×10¹¹ total cells of the Prevotella bacteria (e.g., total dose of a tablet or plurality of tablets).

In some embodiments, the tablet comprises about 9.6×10¹¹ total cells of the Prevotella bacteria (e.g., total dose of a tablet or plurality of tablets).

In some embodiments, the tablet comprises about 12.8×10¹¹ total cells of the Prevotella bacteria (e.g., total dose of a tablet or plurality of tablets).

In some embodiments, the tablet comprises about 16×10¹¹ total cells of the Prevotella bacteria (e.g., total dose of a tablet or plurality of tablets).

In some embodiments, each tablet comprises about 8×10¹⁰ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 8×10¹⁰ total cells) is administered, e.g., once or twice daily to a subject. In some embodiments, 2 tablets (e.g., each comprising about 8×10¹⁰ total cells) are administered, e.g., once or twice daily to a subject. In some embodiments, 4 tablets (e.g., each comprising about 8×10¹⁰ total cells) are administered, e.g., once or twice daily to a subject. In some embodiments, 5 tablets (e.g., each comprising about 8×10¹⁰ total cells) are administered, e.g., once or twice daily to a subject. In some embodiments, 10 tablets (e.g., each comprising about 8×10¹⁰ total cells) are administered, e.g., once or twice daily to a subject.

In some embodiments, each tablet comprises about 1.6×10¹¹ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 1.6×10¹¹ total cells) is administered, e.g., once or twice daily to a subject. In some embodiments, 2 tablets (e.g., each comprising about 1.6×10¹¹ total cells) are administered, e.g., once or twice daily to a subject. In some embodiments, 4 tablets (e.g., each comprising about 1.6×10¹¹ total cells) are administered, e.g., once or twice daily to a subject. In some embodiments, 5 tablets (e.g., each comprising about 1.6×10¹¹ total cells) are administered, e.g., once or twice daily to a subject. In some embodiments, 10 tablets (e.g., each comprising about 1.6×10¹¹ total cells) are administered, e.g., once or twice daily to a subject.

In some embodiments, each tablet comprises about 3.2×10¹¹ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 3.2×10¹¹ total cells) is administered, e.g., once or twice daily to a subject. In some embodiments, 2 tablets (e.g., each comprising about 3.2×10¹¹ total cells) are administered, e.g., once or twice daily to a subject. In some embodiments, 4 tablets (e.g., each comprising about 3.2×10¹¹ total cells) are administered, e.g., once or twice daily to a subject. In some embodiments, 5 tablets (e.g., each comprising about 3.2×10¹¹ total cells) are administered, e.g., once or twice daily to a subject. In some embodiments, 10 tablets (e.g., each comprising about 3.2×10¹¹ total cells) are administered, e.g., once or twice daily to a subject.

In some embodiments, the solid dosage form comprises a mini-tablet. In some embodiments, the mini-tablet is enteric coated. In some embodiments, the mini-tablet is from 1 mm to 4 mm in diameter. In some embodiments, the mini-tablet (e.g., enteric coated mini-tablet) is a 1 mm mini-tablet, 1.5 mm mini-tablet, 2 mm mini-tablet, 3 mm mini-tablet, or 4 mm mini-tablet. In some embodiments, the solid dosage form comprises mini-tablets that comprise about 8×10¹⁰ total cells of the Prevotella bacteria (e.g., total dose of a plurality of mini-tablets). In some embodiments, the solid dosage form comprises mini-tablets that comprise about 1.6×10′ total cells of the Prevotella bacteria (e.g., total dose of a plurality of mini-tablets). In some embodiments, the solid dosage form comprises mini-tablets that comprise about 3.2×10¹¹ total cells of the Prevotella bacteria (e.g., total dose of a plurality of mini-tablets). In some embodiments, the solid dosage form comprises mini-tablets that comprise about 8×10¹¹ total cells of the Prevotella bacteria (e.g., total dose of a plurality of mini-tablets). In some embodiments, the solid dosage form comprises mini-tablets that comprise about 9.6×10¹¹ total cells of the Prevotella bacteria (e.g., total dose of a plurality of mini-tablets). In some embodiments, the solid dosage form comprises mini-tablets that comprise about 12.8×10¹¹ total cells of the Prevotella bacteria (e.g., total dose of a plurality of mini-tablets). In some embodiments, the solid dosage form comprises mini-tablets that comprise about 16×10¹¹ total cells of the Prevotella bacteria (e.g., total dose of a plurality of mini-tablets). In some embodiments, the Prevotella bacteria in the mini-tablets are lyophilized.

In some embodiments, the mini-tablets (e.g., enteric coated mini-tablets) are contained in a capsule. In some embodiments, the capsule is a size 00, size 0, size 1, size 2, size 3, size 4, or size 5 capsule. In some embodiments, the capsule comprises a non-enteric coating (e.g., gelatin or HPMC) (e.g., is coated with a non-enteric coating). In some embodiments, the capsule comprises a non-enteric coating. In some embodiments, the capsule comprises gelatin. In some embodiments, the capsule comprises HPMC.

In some embodiments, the mini-tablets (e.g., enteric coated mini-tablets) that comprise about 8×10¹⁰ total cells of the Prevotella bacteria are contained in a capsule(s).

In some embodiments, the mini-tablets (e.g., enteric coated mini-tablets) that comprise about 1.6×10¹¹ total cells of the Prevotella bacteria are contained in a capsule(s).

In some embodiments, the mini-tablets (e.g., enteric coated mini-tablets) that comprise about 3.2×10¹¹ total cells of the Prevotella bacteria are contained in a capsule(s).

In some embodiments, the mini-tablets (e.g., enteric coated mini-tablets) that comprise about 8×10¹¹ total cells of the Prevotella bacteria are contained in a capsule(s).

In some embodiments, the mini-tablets (e.g., enteric coated mini-tablets) that comprise about 9.6×10¹¹ total cells of the Prevotella bacteria are contained in a capsule(s).

In some embodiments, the mini-tablets (e.g., enteric coated mini-tablets) that comprise about 12.8×10¹¹ total cells of the Prevotella bacteria are contained in a capsule(s).

In some embodiments, the mini-tablets (e.g., enteric coated mini-tablets) that comprise about 16×10¹¹ total cells of the Prevotella bacteria are contained in a capsule(s).

In some embodiments, the pharmaceutical composition comprising Prevotella bacteria is prepared as a powder (e.g., for resuspension or for use in a solid dose form (such as a capsule)) or as a solid dose form, such as a tablet, a mini-tablet, a capsule, a pill, or a powder; or a combination of these forms (e.g., mini-tablets comprised in a capsule). The powder can comprise lyophilized bacteria. In some embodiments, the powder further comprises mannitol, magnesium stearate, and/or colloidal silicon dioxide.

In some embodiments, the pharmaceutical composition (e.g., composition of the total dose administered, e.g., once or twice daily) comprises about 8×10¹⁰ total cells of the Prevotella bacteria.

In some embodiments, the pharmaceutical composition (e.g., composition of the total dose administered, e.g., once or twice daily) comprises about 1.6×10¹¹total cells the Prevotella bacteria.

In some embodiments, the pharmaceutical composition (e.g., composition of the total dose administered, e.g., once or twice daily) comprises about 3.2×10¹¹total cells the Prevotella bacteria.

In some embodiments, the pharmaceutical composition (e.g., composition of the total dose administered, e.g., once or twice daily) comprises about 8×10¹¹ total cells of the Prevotella bacteria.

In some embodiments, the pharmaceutical composition (e.g., composition of the total dose administered, e.g., once or twice daily) comprises about 1.6×10¹⁰ to about 8 x 10¹¹ total cells of the Prevotella bacteria.

In some embodiments, the pharmaceutical composition (e.g., composition of the total dose administered, e.g., once or twice daily) comprises about 1.6×10¹⁰ to about 1.6×10¹¹ total cells of the Prevotella bacteria.

In some embodiments, the pharmaceutical composition (e.g., composition of the total dose administered, e.g., once or twice daily) comprises about 1.6×10¹⁰ to about 16×10¹¹ total cells of the Prevotella bacteria.

In some embodiments, the pharmaceutical composition (e.g., composition of the total dose administered, e.g., once or twice daily) comprises about 8×10¹⁰ to about 8×10¹¹ total cells of the Prevotella bacteria.

In some embodiments, the pharmaceutical composition (e.g., composition of the total dose administered, e.g., once or twice daily) comprises about 1.6×10¹¹to about 8×10¹¹ total cells of the Prevotella bacteria.

In some embodiments, the pharmaceutical composition (e.g., composition of the total dose administered, e.g., once or twice daily) comprises about 9.6×10¹¹total cells of Prevotella histicola , e.g., of Prevotella Strain B 50329.

In some embodiments, the pharmaceutical composition (e.g., composition of the total dose administered, e.g., once or twice daily) comprises about 12.8×10¹¹total cells of Prevotella histicola , e.g., of Prevotella Strain B 50329.

In some embodiments, the pharmaceutical composition (e.g., composition of the total dose administered, e.g., once or twice daily) comprises about 16×10¹¹total cells of Prevotella histicola , e.g., of Prevotella Strain B 50329.

In some embodiments, the pharmaceutical composition (e.g., composition of the total dose administered, e.g., once or twice daily) comprises about 9.6×10¹¹to about 16×10¹¹ total cells of Prevotella histicola , e.g., of Prevotella Strain B 50329.

In some embodiments, the pharmaceutical composition (e.g., composition of the total dose administered, e.g., once or twice daily) comprises about 9.6×10¹¹to about 12.8×10¹¹ total cells of Prevotella histicola , e.g., of Prevotella Strain B 50329.

In some embodiments, the pharmaceutical composition (e.g., composition of the total dose administered, e.g., once or twice daily) comprises about 12.8×10¹¹to about 16×10¹¹ total cells of Prevotella histicola , e.g., of Prevotella Strain B 50329.

In some embodiments, 1, 2, 3, 4, 5, 6, 7, 8, 9, or 10 solid dosage forms are administered, e.g., once or twice daily to a subject.

In some embodiments, 1 solid dosage form (e.g., tablet or capsule) is administered (e.g., is for administration) per day, wherein the solid dosage form comprises a dose of bacteria of about 8×10¹⁰ total cells. In some embodiments, 2 solid dosage forms (e.g., tablets or capsules) are administered (e.g., are for administration) per day, wherein the solid dosage form (e.g., each solid dose form) comprises a dose of bacteria of about 8×10¹⁰ total cells. In some embodiments, 3 solid dosage forms (e.g., tablets or capsules) are administered (e.g., are for administration) per day, wherein the solid dosage form comprises a dose of bacteria of about 8×10¹⁰ total cells. In some embodiments, 4 solid dosage forms (e.g., tablets or capsules) are administered (e.g., are for administration) per a day, wherein the solid dosage form comprises a dose of bacteria of about 8×10¹⁰ total cells. In some embodiments, 5 solid dosage forms (e.g., tablets or capsules) are administered (e.g., are for administration) per a day, wherein the solid dosage form comprises a dose of bacteria of about 8×10¹⁰ total cells. In some embodiments, 6 solid dosage forms (e.g., tablets or capsules) are administered (e.g., are for administration) per a day, wherein the solid dosage form comprises a dose of bacteria of about 8×10¹⁰ total cells. In some embodiments, 8 solid dosage forms (e.g., tablets or capsules) are administered (e.g., are for administration) per a day, wherein the solid dosage form comprises a dose of bacteria of about 8×10¹⁰ total cells. In some embodiments, 10 solid dosage forms (e.g., tablets or capsules) are administered (e.g., are for administration) per a day, wherein the solid dosage form comprises a dose of bacteria of about 8×10¹⁰ total cells.

In some embodiments, 1 solid dosage form (e.g., tablet or capsule) is administered (e.g., is for administration) per day, wherein the solid dosage form comprises a dose of bacteria of about 1.6×10¹¹ total cells. In some embodiments, 2 solid dosage forms (e.g., tablets or capsules) are administered (e.g., are for administration) per day, wherein the solid dosage form (e.g., each solid dose form) comprises a dose of bacteria of about 1.6×10¹¹ total cells. In some embodiments, 3 solid dosage forms (e.g., tablets or capsules) are administered (e.g., are for administration) per day, wherein the solid dosage form comprises a dose of bacteria of about 1.6×10¹¹ total cells. In some embodiments, 4 solid dosage forms (e.g., tablets or capsules) are administered (e.g., are for administration) per a day, wherein the solid dosage form comprises a dose of bacteria of about 1.6×10¹¹ total cells. In some embodiments, 5 solid dosage forms (e.g., tablets or capsules) are administered (e.g., are for administration) per a day, wherein the solid dosage form comprises a dose of bacteria of about 1.6×10¹¹ total cells. In some embodiments, 6 solid dosage forms (e.g., tablets or capsules) are administered (e.g., are for administration) per a day, wherein the solid dosage form comprises a dose of bacteria of about 1.6×10¹¹ total cells. In some embodiments, 8 solid dosage forms (e.g., tablets or capsules) are administered (e.g., are for administration) per a day, wherein the solid dosage form comprises a dose of bacteria of about 1.6×10¹¹ total cells. In some embodiments, 10 solid dosage forms (e.g., tablets or capsules) are administered (e.g., are for administration) per a day, wherein the solid dosage form comprises a dose of bacteria of about 1.6×10¹¹ total cells.

In some embodiments, 1 solid dosage form (e.g., tablet or capsule) is administered (e.g., is for administration) per day, wherein the solid dosage form comprises a dose of bacteria of about 3.2×10¹¹ total cells. In some embodiments, 2 solid dosage forms (e.g., tablets or capsules) are administered (e.g., are for administration) per day, wherein the solid dosage form (e.g., each solid dose form) comprises a dose of bacteria of about 3.2×10¹¹ total cells. In some embodiments, 3 solid dosage forms (e.g., tablets or capsules) are administered (e.g., are for administration) per day, wherein the solid dosage form comprises a dose of bacteria of about 3.2×10¹¹ total cells. In some embodiments, 4 solid dosage forms (e.g., tablets or capsules) are administered (e.g., are for administration) per a day, wherein the solid dosage form comprises a dose of bacteria of about 3.2×10¹¹ total cells. In some embodiments, 5 solid dosage forms (e.g., tablets or capsules) are administered (e.g., are for administration) per a day, wherein the solid dosage form comprises a dose of bacteria of about 3.2×10¹¹ total cells. In some embodiments, 6 solid dosage forms (e.g., tablets or capsules) are administered (e.g., are for administration) per a day, wherein the solid dosage form comprises a dose of bacteria of about 3.2×10¹¹ total cells. In some embodiments, 8 solid dosage forms (e.g., tablets or capsules) are administered (e.g., are for administration) per a day, wherein the solid dosage form comprises a dose of bacteria of about 3.2×10¹¹ total cells. In some embodiments, 10 solid dosage forms (e.g., tablets or capsules) are administered (e.g., are for administration) per a day, wherein the solid dosage form comprises a dose of bacteria of about 3.2×10¹¹ total cells. For clarity, about 3.2×10¹¹ total cells includes total cell counts within ±5% of 3.2×10¹¹ total cells e.g., 3.35×10¹¹ total cells.

In some embodiments, a dose of Prevotella histicola bacteria of about 1.6×10¹¹ to about 1.6×10¹¹ total cells are administered (e.g., are for administration) per day.

In some embodiments, a dose of Prevotella histicola bacteria of about 1.6×10¹⁰ to about 16×10¹¹ total cells are administered (e.g., are for administration) per day. [98] In some embodiments, a dose of Prevotella histicola bacteria of about 8×10¹⁰ to about 8×10¹¹ total cells are administered (e.g., are for administration) per day. [99] In some embodiments, a dose of Prevotella histicola bacteria of about 1.6×10¹¹ to about 8×10¹¹ total cells are administered (e.g., are for administration) per day.

In some embodiments, a dose of Prevotella histicola bacteria of about 9.6×10¹¹ to about 16×10¹¹ total cells are administered (e.g., are for administration) per day.

In some embodiments, a dose of Prevotella histicola bacteria of about 9.6×10¹¹ to about 12.8×10¹¹ total cells are administered (e.g., are for administration) per day.

In some embodiments, a dose of Prevotella histicola bacteria of about 12.8×10¹¹ to about 16×10¹¹ total cells are administered (e.g., are for administration) per day.

In some embodiments, a dose of Prevotella histicola bacteria of about 1.6×10¹¹ total cells are administered (e.g., are for administration) per day.

In some embodiments, a dose of Prevotella histicola bacteria of about 3.2×10¹¹ total cells are administered (e.g., are for administration) per day.

In some embodiments, a dose of Prevotella histicola bacteria of about 8×10¹¹ total cells are administered (e.g., are for administration) per day.

In some embodiments, a dose of Prevotella histicola bacteria of about 9.6×10¹¹ total cells are administered (e.g., are for administration) per day.

In some embodiments, a dose of Prevotella histicola bacteria of about 12.8×10¹¹ total cells are administered (e.g., are for administration) per day.

In some embodiments, a dose of Prevotella histicola bacteria of about 16×10¹¹ total cells are administered (e.g., are for administration) per day.

In some embodiments, the solid dosage form is a tablet. In some embodiments, the tablet is an enteric coated tablet. In some embodiments, the enteric coated tablet is from 5 mm to 18 mm in diameter. In some embodiments, the tablet comprises about 8×10¹⁰ total cells of the Prevotella bacteria. In some embodiments, the tablet comprises about 1.6×10¹¹ total cells of the Prevotella bacteria. In some embodiments, the tablet comprises about 3.2×10¹¹ total cells of the Prevotella bacteria. In some embodiments, the Prevotella bacteria in the tablet are lyophilized.

In some embodiments, the solid dosage form is a capsule. In some embodiments, the capsule is an enteric coated capsule. In some embodiments, the enteric coated capsule is a size 00, size 0, size 1, size 2, size 3, size 4, or size 5 capsule. In some embodiments, the capsule is a size 0 capsule. In some embodiments, the capsule comprises about 8×10¹⁰ total cells of the Prevotella bacteria. In some embodiments, the capsule comprises about 1.6×10¹¹ total cells of the Prevotella bacteria. In some embodiments, the capsule comprises about 3.2×10¹¹ total cells of the Prevotella bacteria. In some embodiments, the Prevotella bacteria in the capsule are lyophilized.

In certain embodiments, provided herein are solid dosage forms comprising the Prevotella bacteria. In some embodiments, the solid dosage form is a tablet, e.g., an enteric coated tablet. In some embodiments, the solid dosage form is a mini-tablet, e.g., an enteric coated mini-tablet. In some embodiments, the solid dosage form is a capsule, e.g., an enteric coated capsule. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P or Eudragit L30-D55).

In some embodiments, the pharmaceutical composition comprising Prevotella bacteria is prepared as a powder. The powder can comprise lyophilized bacteria. In some embodiments, the powder further comprises mannitol, magnesium stearate, and/or colloidal silicon dioxide. In some embodiments, the pharmaceutical composition comprises a powder comprising Prevotella bacteria. In some embodiments, the powder comprising Prevotella bacteria (e.g., at a dose provided herein) is resuspended (e.g., in a liquid such as a solution, buffer, water or other beverage, or a food), e.g., for use in the methods provided herein.

In some embodiments, the pharmaceutical composition is administered orally.

In some embodiments, the administration to the subject once daily. In some embodiments, the pharmaceutical composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the administration to the subject twice daily. In some embodiments, the pharmaceutical composition is administered twice daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the Prevotella histicola strain is administered in a pharmaceutical composition (e.g., a pharmaceutical composition provided herein). In certain embodiments the pharmaceutical composition is a solid dose form provided herein. In some embodiments, the pharmaceutical composition comprises a blend of freeze-dried powder of Prevotella histicola and excipients (e.g. an encapsulated freeze-dried powder of a Prevotella histicola strain provided herein and excipients). In some embodiments, the pharmaceutical composition comprises freeze-dried (e.g., lyophilized) powder of bacteria in a capsule. In some embodiments, the capsule is enteric coated. In some embodiments, the pharmaceutical composition comprises an enteric coated hydroxylpropyl methylcellulose (HPMC) hard capsule. In some embodiments, the pharmaceutical composition comprises a formulation of Prevotella histicola Strain B comprising freeze-dried powder of Prevotella histicola and excipients. In some embodiments, the excipients include mannitol, magnesium stearate and colloidal silicon dioxide. In some embodiments, each capsule contains about 8.0×10¹⁰ cells of a Prevotella histicola strain provided herein (e.g., Prevotella histicola Strain B). In some embodiments, 1, 2, 3, 4, 5, 6, 7, 8, 9, or 10 powder-containing capsules are administered to a subject daily. In some embodiments, 1, 2, 3, 4, 5, 6, 7, 8, 9, or 10 powder-containing capsules (e.g., each containing about 8.0×10¹⁰ cells of a Prevotella histicola strain provided herein (e.g., Prevotella histicola Strain B)) are administered to a subject once daily. In some embodiments, 1, 2, 3, 4, 5, 6, 7, 8, 9, or 10 powder-containing capsules (e.g., each containing about 8.0×10¹⁰ cells of a Prevotella histicola strain provided herein (e.g., Prevotella histicola Strain B)) are administered to a subject twice daily. In some embodiments, 2 powder-containing capsules are administered to the subject daily. In some embodiments, 1 powder-containing capsule is administered to the subject daily. In some embodiments, each powder-containing capsule contains about 8.0×10¹⁰ cells of a Prevotella histicola strain provided herein (e.g., Prevotella histicola Strain B). In some embodiments, 1, 2, 3, 4, 5, 6, 7, 8, 9, or 10 powder-containing capsules are administered to a subject daily. In some embodiments, 2 powder-containing enteric coated capsules (e.g., each containing about 8.0×10¹⁰ cells of a Prevotella histicola strain provided herein (e.g., Prevotella histicola Strain B)) are administered to the subject daily. In some embodiments, 4 powder-containing enteric coated capsules (e.g., each containing about 8.0×10¹⁰ cells of a Prevotella histicola strain provided herein (e.g., Prevotella histicola Strain B)) are administered to the subject daily. In some embodiments, 2 powder-containing enteric coated capsules (e.g., each containing about 8.0×10¹⁰ cells of a Prevotella histicola strain provided herein (e.g., Prevotella histicola Strain B)) are administered to the subject once daily. In some embodiments, 2 powder-containing enteric coated capsules (e.g., each containing about 8.0×10¹⁰ cells of a Prevotella histicola strain provided herein (e.g., Prevotella histicola Strain B)) are administered to the subject twice daily. In some embodiments, 2 powder-containing enteric coated capsules (e.g., each containing about 8.0×10¹⁰ cells of a Prevotella histicola strain provided herein (e.g., Prevotella histicola Strain B)) are administered to the subject twice daily (e.g., for 1-7 days, 3 days, 7 days, 10 days, or 14 days), and then 2 powder-containing enteric coated capsules (e.g., each containing about 8.0×10¹⁰ cells of a Prevotella histicola strain provided herein (e.g., Prevotella histicola Strain B)) are administered to the subject once daily, e.g., for the duration of the treatment period (e.g., up to 14 days of total treatment). In some embodiments, 1 powder-containing enteric coated capsule (e.g., containing about 8.0×10¹⁰ cells of a Prevotella histicola strain provided herein (e.g., Prevotella histicola Strain B)) is administered to the subject daily.

In some embodiments, the pharmaceutical composition is formulated as multiple enteric-coated mini-tablets of Prevotella histicola drug product filled into capsules. In some embodiments, the pharmaceutical composition is formulated as multiple enteric-coated mini-tablets of Prevotella histicola drug product filled into capsules, e.g., HPMC capsules (MICs). In some embodiments, the pharmaceutical composition comprises excipients (e.g., pharmaceutically acceptable excipients). In some embodiments, the pharmaceutical composition comprises mannitol, colloidal silicon dioxide, hydroxypropyl cellulose, crospovidone, and magnesium stearate. In some embodiments, each capsule contains about 8.0×10¹⁰ cells of a Prevotella histicola strain provided herein (e.g., Prevotella histicola Strain B). In some embodiments, 1, 2, 3, 4, 5, 6, 7, 8, 9, or 10 capsules are administered to a subject daily. In some embodiments, 2 capsules are administered to the subject daily. In some embodiments, 1 capsule is administered to the subject daily. In some embodiments, each MIC contains about 8.0×10¹⁰ cells of a Prevotella histicola strain provided herein (e.g., Prevotella histicola Strain B). In some embodiments, 1, 2, 3, 4, 5, 6, 7, 8, 9, or 10 MICs are administered to a subject daily. In some embodiments, 2 MICs are administered to the subject daily. In some embodiments, 1 MIC is administered to the subject daily. In some embodiments, each capsule contains about 8.0×10¹⁰ cells of a Prevotella histicola strain provided herein (e.g., Prevotella histicola Strain B). In some embodiments, each MIC contains about 1.6×10¹¹ cells of a Prevotella histicola strain provided herein (e.g., Prevotella histicola Strain B). In some embodiments, 1, 2, 3, 4, 5, 6, 7, 8, 9, or 10 MICs are administered to a subject daily. In some embodiments, 2 MICs are administered to the subject daily. In some embodiments, 1 MIC is administered to the subject daily. In some embodiments, each capsule contains about 1.6×10¹¹ cells of a Prevotella histicola strain provided herein (e.g., Prevotella histicola Strain B). In some embodiments, each MIC contains about 3.2×10¹¹ cells of a Prevotella histicola strain provided herein (e.g., Prevotella histicola Strain B). In some embodiments, 1, 2, 3, 4, 5, 6, 7, 8, 9, or 10 MICs are administered to a subject daily. In some embodiments, 2 MICs are administered to the subject daily. In some embodiments, 1 MIC is administered to the subject daily. In some embodiments, each capsule contains about 3.2×10¹¹ cells of a Prevotella histicola strain provided herein (e.g., Prevotella histicola Strain B).

In some embodiments, the Prevotella histicola strain is a strain comprising at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is the Prevotella histicola Strain B (NRRL accession number B 50329).

In some aspects, the disclosure provides use of a Prevotella histicola strain provided herein and/or a pharmaceutical composition (e.g., a pharmaceutical composition and/or a solid dosage form) described herein (e.g., in an amount described herein) for the preparation of a medicament for the performance of a therapeutic method provided herein. In some aspects, the disclosure provides a Prevotella histicola strain provided herein and/or a pharmaceutical composition (e.g., a pharmaceutical composition and/or a solid dosage form) described herein (e.g., in an amount described herein) for use in the performance of a therapeutic method provided herein.

In some embodiments, the subject treated according to the methods provided herein has an IL-8-mediated disease or condition. In certain embodiments, the IL-8 mediated disease or condition comprises Severe Acute Respiratory Syndrome (SARS), influenza, respiratory syncytial viral infection, atherosclerosis, melanoma, ovarian carcinoma, lung cancer, prostate cancer, gastric carcinoma, breast cancer, head-and-neck cancer, colon cancer, colitis-associated cancer, kidney cancer, pancreatic cancer, Crohn's disease (CD), Ulcerative Colitis (UC), Ischemia-Reperfusion injury (IRI), acute lung injury, asthma, chronic obstructive pulmonary disease (COPD), cystic fibrosis (CF), pulmonary fibrosis, multiple sclerosis, psoriasis, atopic dermatitis, rheumatoid arthritis, crescentic glomerulonephritis, IgA nephropathy, membranoproliferative glomerulonephritis, lupus nephritis, or membranous nephropathy, alcoholic hepatitis, or HIV-associated neurocognitive disorder. In certain embodiments, the IL-8 mediated disease or condition comprises Severe Acute Respiratory Syndrome (SARS), influenza, or a respiratory syncytial viral infection. In certain embodiments, the IL-8 mediated disease or condition comprises a coronavirus infection (e.g., MERS, SARS (such as SARS-CoV-2)). In certain embodiments, the IL-8 mediated disease or condition comprises SARS-CoV-2 infection. In certain embodiments, the IL-8 mediated disease or condition is COVID-19.

In some embodiments, the subject treated according to the methods provided herein has an IL-6 mediated disease or condition. In certain embodiments, the IL-6 mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-6 mediated disease or condition comprises Severe Acute Respiratory Syndrome (SARS), influenza, or a respiratory syncytial viral infection. In certain embodiments, the IL-6 mediated disease or condition comprises a coronavirus infection (e.g., MERS, SARS (such as SARS-CoV-2)). In certain embodiments, the IL-6 mediated disease or condition comprises SARS-CoV-2 infection. In certain embodiments, the IL-6 mediated disease or condition is COVID-19.

In some embodiments, the subject treated according to the methods provided herein has an IL-1β mediated disease or condition. In certain embodiments, the IL-1β mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-1β mediated disease or condition comprises Severe Acute Respiratory Syndrome (SARS), influenza, or a respiratory syncytial viral infection. In certain embodiments, the IL-1β mediated disease or condition comprises a coronavirus (e.g., SARS-CoV-2). In certain embodiments, the IL-1β mediated disease or condition comprises SARS-CoV-2 infection. In certain embodiments, the IL-1β mediated disease or condition is COVID-19.

In some embodiments, the subject treated according to the methods provided herein has a TNFα mediated disease or condition. In some embodiments, the TNFα mediated disease or condition is Severe Acute Respiratory Syndrome (SARS), influenza, respiratory syncytial viral infection, rheumatoid arthritis, juvenile chronic arthritis, Crohn's disease (CD), Ulcerative Colitis (UC), ankylosing spondylitis, psoriasis, multiple sclerosis, atherosclerosis, myocardial infarction, heart failure, myocarditis, cardiac allograft rejection, asthma, ischemic renal injury, renal transplant rejection, glomerulonephritis, or inflammatory eye disease. In some embodiments, the TNFa mediated disease or condition is Severe Acute Respiratory Syndrome (SARS), influenza, or a respiratory syncytial viral infection. In certain embodiments, the TNFα mediated disease or condition comprises a coronavirus infection (e.g., MERS, SARS (such as SARS-CoV-2)). In certain embodiments, the TNFα mediated disease or condition comprises SARS-CoV-2 infection. In certain embodiments, the TNFα mediated disease or condition is COVID-19.

In some embodiments, the subject treated according to the methods provided herein has secondary hemophagocytic lymphohistiocytosis (sHLH).

In some embodiments, the subject treated according to the methods provided herein has a COVID-Related Complication (CRC). In some embodiments, the CRC comprises acute respiratory distress syndrome (ARDS), arrhythmia, shock, acute kidney injury, acute cardiac injury, liver dysfunction and/or secondary infection. In some embodiments, the subject treated according to the methods provided herein has ARDS.

In some embodiments, the methods provided herein further comprise administering to the subject an additional therapy. In some embodiments, the additional therapy comprises the standard of care for the disease being treated (e.g., a coronavirus infection, such as a MERS or SARS (e.g., SARS-CoV-2) infection). In some embodiments, the methods provided herein further comprise administering to the subject an antiviral medication. In some embodiments, the methods provided herein further comprise administering to the subject an antiviral medication such as ribavirin, neuraminidase inhibitor, protease inhibitor, recombinant interferons, antibodies, oseltamivir, zanamivir, peramivir or baloxavir marboxil. In some embodiments, the method further comprises administering to the subject hydroxychloroquine and/or chloroquine. In some embodiments, the method further comprises administering to the subject remdesivir. In some embodiments, the method further comprises administering to the subject an angiotensin-converting enzyme (ACE) inhibitor. In some embodiments, the method further comprises administering to the subject an angiotensin-converting enzyme 2 (ACE2) inhibitor. In some embodiments, the method further comprises administering to the subject plasma from a subject who has recovered from infection by the same virus that is infecting the subject (e.g., plasma from a subject who has recovered from SARS-CoV-2 infection) (e.g., convalescent plasma therapy). In some embodiments, the method further comprises administering (e.g., orally administering) to the subject an anti-inflammatory agent such as an NSAID or an anti-inflammatory steroid. In some embodiments, the method further comprises administering (e.g., orally or intravenously administering) to the subject a corticosteroid such as dexamethasone, prednisone, methylprednisolone, or hydrocortisone. In some embodiments, the method further comprises administering (e.g., orally or intravenously administering) to the subject dexamethasone. In some embodiments, the method further comprises administering to the subject IFN-β1a (e.g., by inhalation). In some embodiments, the method further comprises administering to the subject SNG001 (IFN-β1a for Nebulisation).

In some embodiments, the method further comprises administering to the subject an antibody specific for IL-6 and/or the IL-6 receptor. In some embodiments, the method comprises administering to the subject tocilizumab (Actemra®). In some embodiments, the method comprises administering to the subject sarilumab (Kevzara®).

In some embodiments, the method further comprises administering to the subject a monoclonal antibody treatment. In some embodiments, the method further comprises administering to the subject a monoclonal antibody treatment such as bamlanivimab, casirivimab, or imdevimab, or a combination thereof, e.g., a combination of casirivimab and imdevimab. In some embodiments, the additional therapy can comprise a monoclonal antibody treatment such as bamlanivimab, casirivimab, or imdevimab, or a combination thereof, e.g., a combination of casirivimab and imdevimab. In some embodiments, the method further comprises administering to the subject a monoclonal antibody treatment such as bamlanivimab or etesevimab, or a combination of bamlanivimab or etesevimab.

In some embodiments, the additional therapy can comprise budesonide, e.g., inhaled budesonide.

In some embodiments, the method further comprises administering to the subject baricitinib.

In some embodiments, the method further comprises administering to the subject baricitinib in combination with remdesivir.

In some embodiments, the method further comprises administering to the subject an anticoagulation drug, such as heparin or enoxaparin (e.g., a low-dose thereof).

In some embodiments, the method further comprises administering to the subject vitamin D.

In some embodiments, the method further comprises administering to the subject plitidepsin (also referred to as dehydrodidemnin B) (e.g., marketed as Aplidin).

In some embodiments, the method further comprises administering to the subject ivermectin.

In certain aspects, provided herein is a method of identifying a subject at risk for increased severity of a disease or condition (e.g., increased symptom severity associated with a viral infection and/or increased symptom severity associated with an IL8, IL-6, IL-1β and/or TNFα mediated disease or condition) comprising determining expression levels IL-8, IL-6, IL-1β, and/or TNFα in a sample from the subject (e.g., a blood sample contacted with LPS), wherein elevated expression of IL-8, IL-6, IL-1β, and/or TNFα indicates that the subject is at risk for increased severity of the disease or condition. Expression can be elevated as compared to a standard, such as the mean or median level of expression of the cytokine in a cohort of healthy subjects or a cohort of subjects who have not been diagnosed with a viral infection or historical levels. In some embodiments, the method further comprises treating the subject for the disease or condition (e.g., using a method provided herein). In some embodiments, the disease or condition comprises cytokine storm syndrome (cytokine release syndrome) (e.g., a cytokine storm resulting from a viral infection, such as a SARS-CoV-2 infection). In some embodiments, the disease or condition comprises Severe Acute Respiratory Syndrome (SARS), influenza, or a respiratory syncytial viral infection. In certain embodiments, the disease or condition comprises a coronavirus infection (e.g., MERS, SARS (such as SARS-CoV-2)). In certain embodiments, the disease or condition comprises SARS-CoV-2 infection. In some embodiments, the disease or condition is COVID-19.

### BRIEF DESCRIPTION OF THE DRAWINGS

FIG. 1 shows a waterfall plot illustrating the percent change in IL-8 expression by subjects after 28 days of treatment with Prevotella histicola Strain B (right) or placebo (left).

FIG. 2 shows a waterfall plot illustrating the percent change in IL-6 expression by subjects after 28 days of treatment with Prevotella histicola Strain B (right) or placebo (left).

FIG. 3 shows a waterfall plot illustrating the percent change in TNFa expression by subjects after 28 days of treatment with Prevotella histicola Strain B (right) or placebo (left).

FIG. 4 shows a waterfall plot illustrating the percent change in IL-1β expression by subjects after 28 days of treatment with Prevotella histicola Strain B (right) or placebo (left).

FIG. 5 is two panels showing IFNα (left panel) and IFNβ (right panel) levels in spleen cells removed from animals treated with Prevotella histicola Strain B (“Strain B”) or dexamethasone or a combination thereof. The effect of the treatments on virally-induced production of these interferons was mimicked by treating the cells with poly (I:C).

FIG. 6 is two panels showing IL6 (left panel) and TNFα (right panel) levels in spleen cells removed from animals treated with Prevotella histicola Strain B (“Strain B”) or dexamethasone or a combination thereof.

FIG. 7 is a graph showing the effects of Prevotella histicola Strain B (“Strain B”) or dexamethasone or a combination thereof on ear inflammation in a KLH DTH model.

### DETAILED DESCRIPTION

General

In certain aspects, provided herein is a method of reducing IL-8, IL-6, IL-1β, and/or TNFα expression levels in a subject in need thereof, comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329).

In certain aspects, provided herein is a method of treating a viral infection in a subject comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329). In some embodiments, the viral infection is a coronavirus infection, an influenza infection, and/or a respiratory syncytial virus infection. In some embodiments, the viral infection is a SARS-CoV-2 infection.

In certain aspects, provided herein is a method of treating COVID-19 in a subject comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329).

In certain aspects, provided herein is a method of treating and/or reducing the severity of cytokine storm syndrome (cytokine release syndrome) (e.g., a cytokine storm resulting from a viral infection, such as a SARS-CoV-2 infection) in a subject comprising administering to the subject a Prevotella histicola strain comprising at least 99% genomic, 16S and/or CRISPR sequence identity to the nucleotide sequence of the Prevotella histicola Strain B (NRRL accession number B 50329). In some embodiments, the Prevotella histicola strain is administered in a pharmaceutical composition and/or a solid dosage form. In some embodiments, the Prevotella histicola strain is Prevotella histicola Strain B (NRRL accession number B 50329).

In certain embodiments, the therapeutic effects of these orally delivered medicines come from their action on pattern recognition receptors on immune cells in the lining of the small intestine. These cells, in turn, modulate immune cells circulating throughout the body. The medicines are microbes, but do not target the microbiome. In some embodiments, the microbes do not colonize or persist in the gut and do not modify the colonic microbiome. In some embodiments, they are gut-restricted.

Prevotella histicola Strain B (NRRL accession number B 50329) has recently completed a series of cohorts in a phase lb study in human volunteers and patients with psoriasis.

The primary endpoints were safety and tolerability. Prevotella histicola Strain B (NRRL accession number B 50329) has a placebo-like profile, consistent with the lack of systemic absorption. There was no persistence beyond the 28 day daily dosing period and no modification of the colonic microbiome by 16S RNA sequencing of patient stool samples.

Two cohorts of patients with mild-to-moderate psoriasis were treated with a low and high dose of Prevotella histicola Strain B (NRRL accession number B 50329) daily for 28 days. The lower dose was estimated by allometric scaling of the just-maximally effective dose in mouse inflammation models. The high dose was 5× higher. There were 12 and 18 patients respectively in these cohorts. The cohorts were recruited independently and sequentially with internal placebo control with 2:1 randomization of active:placebo.

Clinical symptoms and biomarkers of systemic inflammation were the pharmacodynamic endpoints.

At both doses there was a clear clinical response measured by PASI and lesional severity score. In the face of the short duration of treatment and small numbers of subjects, a clear and reproducible treatment effect was seen.

The biomarkers of systemic inflammation were determined by stimulation ex vivo with lipopolysaccharide (LPS) of whole blood samples taken at baseline and after 28 days of treatment. LPS is a potent activator of the myeloid compartment of innate immunity and inflammation, especially on human cells. Reduction of the production of inflammatory cytokines and chemokines in these cultures is a measure of the state of systemic inflammatory activation.

In both cohorts of psoriasis patients there was an overall reduction in inflammatory biomarkers. This is consistent with ex vivo analysis of preclinical models where a pronounced effect is the coordinated down-regulation of multiple inflammatory pathways. Cytokines and chemokines associated with TH1, TH2 and TH17 responses are all beneficially affected.

As disclosed herein, this immune connectivity between the small intestine and the rest of the body suggests a possible therapeutic approach for diseases in which the host inflammatory response becomes overwhelming, such as bacterial septic shock and morbidity and mortality associated with viral infections, including flu and SARS-CoV-2.

This was reinforced by a detailed analysis of the systemic inflammatory biomarkers which showed in patients (e.g., subjects) that there was a marked down-regulation of the production of interleukin-8 (IL-8 or IL8) and interleukin-6 (IL-6 or IL6) in response to LPS stimulation of whole blood from human subjects who had been treated with Prevotella histicola Strain B (NRRL accession number B 50329). There were similar directional effects on TNFα (TNFα) and IL1b (IL-1β or IL1β).

These cytokines and chemokines are key players in host pathology in a range of infectious diseases, including viral infections and severe acute respiratory syndrome. This human multi-cytokine pharmacology of Prevotella histicola Strain B (NRRL accession number B 50329), together with its clinical safety profile, make it an important experimental agent for the control of host responses to infection.

In certain embodiments, Prevotella histicola Strain B (NRRL accession number B 50329) is unique as an anti-inflammatory agent for several reasons: (a) novel mechanism of action in the small intestine; (b) breadth of anti-inflammatory effects; (c) clinical safety and tolerability profile; (d) ease of oral administration; and/or (e) manufacturability at large scale and reasonable cost of drug substance.

As disclosed herein, Prevotella histicola Strain B (NRRL accession number B 50329) is useful for the down-regulation of host responses to viral infection.

### Definitions

The term “about” when used before a numerical value indicates that the value may vary within a reasonable range, such as within ±10%, ±5% or ±1% of the stated value.

“Administration” broadly refers to a route of administration of a composition to a subject. Examples of routes of administration include oral administration, rectal administration, topical administration, inhalation (nasal) or injection. Administration by injection includes intravenous (IV), intramuscular (IM), intratumoral (IT) and subcutaneous (SC) administration. The pharmaceutical compositions described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), intravesical, intrapulmonary, intraduodenal, intragastrical, and intrabronchial. In preferred embodiments, the pharmaceutical compositions described herein are administered orally, rectally, intratumorally, topically, intravesically, by injection into or adjacent to a draining lymph node, intravenously, by inhalation or aerosol, or subcutaneously. In preferred embodiments, the pharmaceutical composition or solid dosage form described herein is administered orally.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include number of immune cells (e.g., of a particular immune cell type), bacterial cells, stromal cells, myeloid derived suppressor cells, fibroblasts, metabolites, and level of cytokines (e.g., a pro-inflammatory cytokine).

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10^3 fold, 10^ fold, 10^5 fold, 10¹\6 fold, and/or 10¹\7 fold greater after treatment when compared to a pre-treatment state. Properties that may be increased include number of immune cells (e.g., of a particular immune cell type), bacterial cells, stromal cells, myeloid derived suppressor cells, fibroblasts, metabolites, and level of cytokines (e.g., a pro-inflammatory cytokine).

The terms “subject” or “patient” refers to any animal. A subject or a patient described as “in need thereof” refers to one in need of a treatment for a disease. Mammals (i.e., mammalian animals) include humans, laboratory animals (e.g., primates, rats, mice), livestock (e.g., cows, sheep, goats, pigs), and household pets (e.g., dogs, cats, rodents). For example, the subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject or patient may be healthy, or may be suffering from (or at increased risk of developing) an immune disorder at any developmental stage or from (or at an increased risk of developing) an infection. In preferred embodiments, the subject is a human. For example, a “subject in need thereof” can be, e.g., a subject who has been diagnosed with a viral infection and/or experiencing a symptom of a viral infection, e.g., a viral infection described herein, a bacterial infection, and/or a subject experiencing a symptom of a cytokine release syndrome, and /or a subject having an exaggerated host cytokine response, e.g., as determined by change from baseline in a cytokine level (such as IL-8, IL-6, IL-1β, and/or TNFa), e.g., at day 4 and/or day 7.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening. For example, “treating” may decrease the level of IL-8, IL-6, IL-1β, and/or TNFα in a subject, e.g., as compared to the level prior to treatment; “treating” may prevent an increase (or cause a decrease) in the level of IL-8, IL-6, IL-1β, and/or TNFα in a subject as compared to a standard, e.g., as compared to the level prior to treatment; “treating” may decrease a clinical factor, such as time on a ventilator or duration of hospitalization as compared to a standard, e.g., as compared to the time or duration in a cohort of subjects who did not receive the treatment.

### Bacteria

Prevotella Histicola. In certain aspects, provided herein are methods of treating a viral infection (or a bacterial septic shock) and pharmaceutical compositions (e.g., a solid dosing form) comprising Prevotella histicola strain provided herein and methods of treating an IL-8, IL-6, IL-1β, and/or TNFα-mediated disease or condition using such Prevotella histicola strains. In some embodiments, the Prevotella strain is a strain of Prevotella histicola . In some embodiments, the Prevotella strain is Prevotella histicola Strain B (NRRL accession number B 50329). In some embodiments, the Prevotella strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic, 16S or CRISPR nucleotide sequence) of the Prevotella histicola Strain B (NRRL accession number B 50329). In some embodiments, the Prevotella strain is Prevotella histicola Strain B (NRRL accession number B 50329).

In certain aspects, provided herein are methods of reducing IL-8, IL-6, IL-1β, and/or TNFα levels in a subject and pharmaceutical compositions (e.g., a solid dosage form) comprising a Prevotella histicola strain provided herein. In some embodiments, the Prevotella strain is a strain of Prevotella histicola . In some embodiments, the Prevotella strain is Prevotella histicola Strain B (NRRL accession number B 50329). In some embodiments, the Prevotella strain is a strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic, 16S or CRISPR nucleotide sequence) of Prevotella histicola Strain B (NRRL accession number B 50329). In some embodiments, the Prevotella strain is Prevotella histicola Strain B (NRRL accession number B 50329).

Applicant represents that the ATCC is a depository affording permanence of the deposit and ready accessibility thereto by the public if a patent is granted. All restrictions on the availability to the public of the material so deposited will be irrevocably removed upon the granting of a patent. The material will be available during the pendency of the patent application to one determined by the Commissioner to be entitled thereto under 37 CFR 1.14 and 35 U.S.C. 122. The deposited material will be maintained with all the care necessary to keep it viable and uncontaminated for a period of at least five years after the most recent request for the furnishing of a sample of the deposited plasmid, and in any case, for a period of at least thirty (30) years after the date of deposit or for the enforceable life of the patent, whichever period is longer. Applicant acknowledges its duty to replace the deposit should the depository be unable to furnish a sample when requested due to the condition of the deposit.

Prevotella histicola Strain B can be cultured according to methods known in the art. For example, Prevotella histicola can be grown in ATCC Medium 2722, ATCC Medium 1490, or other medium using methods disclosed, for example in Caballero et al., 2017. “Cooperating Commensals Restore Colonization Resistance to Vancomycin-Resistant Enterococcus faecium” Cell Host & Microbe 21:592-602, which is hereby incorporated by reference in its entirety.

Bifidobacterium Breve. In certain aspects of the present invention, a Bifidobacterium breve strain is used in place of the Prevotella histicola strain, e.g., in the pharmaceutical compositions, methods, and uses described herein. In some embodiments, the Bifidobacterium breve is the Bifidobacterium breve strain deposited under accession number NCIMB 42380, also referred to as “MRx004” and “MRx4DP0004”. See also, U.S. Patent Pub. No. 2019-0099458, hereby incorporated by reference in its entirety. In some embodiments, the Bifidobacterium breve strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Bifidobacterium breve strain deposited under accession number NCIMB 42380. In some embodiments, the Bifidobacterium breve is lyophilized. In some embodiments, the Bifidobacterium breve comprises live bacteria. In some embodiments, the Bifidobacterium breve daily dose is 4×10⁹ to 4×10¹⁰ colony forming units (CFUs). In some embodiments, the Bifidobacterium breve is taken as 2 capsules, twice a day for 14 days.

### Pharmaceutical Compositions

In certain embodiments, provided herein are pharmaceutical compositions (e.g., solid dosage forms) comprising Prevotella histicola bacteria provided herein.

In some embodiments, the pharmaceutical compositions comprise whole Prevotella histicola bacteria (e.g., live bacteria, killed bacteria, attenuated bacteria).

In some embodiments, the pharmaceutical compositions comprise live Prevotella histicola bacteria.

In some embodiments, the pharmaceutical compositions comprise viable Prevotella histicola bacteria.

In certain embodiments, the pharmaceutical compositions comprise non-viable Prevotella histicola bacteria.

In some embodiments, the pharmaceutical compositions comprise only one strain of bacteria, e.g., Prevotella histicola , e.g., Prevotella Strain B 50329.

In some embodiments, the Prevotella histicola is Prevotella histicola Strain B (NRRL accession number B 50329). In some embodiments, the Prevotella histicola strain is a strain comprising at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B.

In some embodiments, the pharmaceutical composition is formulated as a capsule or a tablet. In some embodiments, the pharmaceutical composition comprises an enteric coating or micro encapsulation. In some embodiments, the pharmaceutical composition is prepared as a capsule. In some embodiments, the capsule is an enteric coated capsule. In some embodiments, the pharmaceutical composition is prepared as a tablet. In some embodiments, the tablet is an enteric coated tablet. In some embodiments, the enteric coating allows release of the pharmaceutical composition in the small intestine, e.g., in the upper small intestine, e.g., in the duodenum.

In some embodiments, the pharmaceutical composition comprises about 50 mg to about 3 g of Prevotella histicola , e.g., of Prevotella Strain B 50329.

In some embodiments, the pharmaceutical composition comprises about 55 mg, about 550 mg, or about 2.76 g of Prevotella histicola , e.g., of Prevotella Strain B 50329.

In some embodiments, the pharmaceutical composition comprises about 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹, 5×10¹¹, 6×10¹¹, 7×10¹¹, 8×10¹¹, 9×10¹¹, 1×10¹², 1.5×10¹² total cells of Prevotella histicola , e.g., of Prevotella Strain B 50329. In some embodiments, the pharmaceutical composition comprises about 8×10¹⁰ total cells of Prevotella histicola , e.g., of Prevotella Strain B 50329. In some embodiments, the pharmaceutical composition comprises about 1.6×10¹¹ total cells of Prevotella histicola , e.g., of Prevotella Strain B 50329. In some embodiments, the pharmaceutical composition comprises about 3.2×10¹¹ total cells of Prevotella histicola , e.g., of Prevotella Strain B 50329. In some embodiments, the pharmaceutical composition comprises about 8×10¹¹ total cells of Prevotella histicola , e.g., of Prevotella Strain B 50329. In some embodiments, the pharmaceutical composition comprises about 9.6×10¹¹ total cells of Prevotella histicola , e.g., of Prevotella Strain B 50329. In some embodiments, the pharmaceutical composition comprises about 12.8×10¹¹ total cells of Prevotella histicola , e.g., of Prevotella Strain B 50329. In some embodiments, the pharmaceutical composition comprises about 16×10¹¹ total cells of Prevotella histicola , e.g., of Prevotella Strain B 50329. In some embodiments, the pharmaceutical composition comprises about 1.6×10¹⁰ to about 1.6×10¹¹ total cells of Prevotella histicola , e.g., of Prevotella Strain B 50329. In some embodiments, the pharmaceutical composition comprises about 1.6×10¹⁰ to about 16×10¹¹ total cells of Prevotella histicola , e.g., of Prevotella Strain B 50329. In some embodiments, the pharmaceutical composition comprises about 8×10¹⁰ to about 8×10¹¹ total cells of Prevotella histicola , e.g., of Prevotella Strain B 50329. In some embodiments, the pharmaceutical composition comprises about 8×10¹⁰ to about 1.6×10¹¹ total cells of Prevotella histicola , e.g., of Prevotella Strain B 50329. In some embodiments, the pharmaceutical composition comprises about 1.6×10¹¹to about 8×10¹¹ total cells of Prevotella histicola , e.g., of Prevotella Strain B 50329. In some embodiments, the pharmaceutical composition comprises about 9.6×10¹¹to about 16×10¹¹ total cells of Prevotella histicola , e.g., of Prevotella Strain B 50329. In some embodiments, the pharmaceutical composition comprises about 9.6×10¹¹to about 12.8×10¹¹ total cells of Prevotella histicola , e.g., of Prevotella Strain B 50329. In some embodiments, the pharmaceutical composition comprises about 12.8×10¹¹to about 16×10¹¹ total cells of Prevotella histicola , e.g., of Prevotella Strain B 50329. Herein, total cells is determined by total cell count (e.g., determined by Coulter counter).

In some embodiments, the pharmaceutical composition comprises about 1.6×10¹⁰ total cells of Prevotella histicola , e.g., of Prevotella Strain B 50329.

In some embodiments, the pharmaceutical composition comprises about 8×10¹⁰ total cells of Prevotella histicola , e.g., of Prevotella Strain B 50329.

In some embodiments, the pharmaceutical composition comprises about 1.6×10¹¹ total cells of Prevotella histicola , e.g., of Prevotella Strain B 50329.

In some embodiments, the pharmaceutical composition comprises about 3.2×10¹¹total cells of Prevotella histicola , e.g., of Prevotella Strain B 50329.

In some embodiments, the pharmaceutical composition comprises about 8×10¹¹total cells of Prevotella histicola , e.g., of Prevotella Strain B 50329.

In some embodiments, the pharmaceutical composition comprises about 1.6×10¹⁰ to about 8×10¹¹ total cells of Prevotella histicola , e.g., of Prevotella Strain B 50329.

In some embodiments, the pharmaceutical composition comprises about 1.6×10¹⁰ to about 1.6×10¹¹ total cells of Prevotella histicola , e.g., of Prevotella Strain B 50329.

In some embodiments, the pharmaceutical composition comprises about 1.6×10¹¹to about 8×10¹¹ total cells of Prevotella histicola , e.g., of Prevotella Strain B 50329.

In some embodiments, the pharmaceutical composition comprises about 8×10¹⁰ to about 8×10¹¹ total cells of Prevotella histicola , e.g., of Prevotella Strain B 50329.

In some embodiments, the pharmaceutical composition, e.g., pharmaceutical composition (e.g., composition of the total dose administered, e.g., once or twice daily) comprises about 9.6×10¹¹ total cells of Prevotella histicola , e.g., of Prevotella Strain B 50329.

In some embodiments, the pharmaceutical composition, e.g., pharmaceutical composition (e.g., composition of the total dose administered, e.g., once or twice daily) comprises about 12.8×10¹¹total cells of Prevotella histicola , e.g., of Prevotella Strain B 50329.

In some embodiments, the pharmaceutical composition, e.g., pharmaceutical composition (e.g., composition of the total dose administered, e.g., once or twice daily) comprises about 16×10¹¹total cells of Prevotella histicola , e.g., of Prevotella Strain B 50329.

In some embodiments, the pharmaceutical composition, e.g., pharmaceutical composition (e.g., composition of the total dose administered, e.g., once or twice daily) comprises about 9.6×10¹¹to about 16×10¹¹ total cells of Prevotella histicola, e.g., of Prevotella Strain B 50329.

In some embodiments, the pharmaceutical composition, e.g., pharmaceutical composition (e.g., composition of the total dose administered, e.g., once or twice daily) comprises about 9.6×10¹¹ to about 12.8×10¹¹ total cells of Prevotella histicola, e.g., of Prevotella Strain B 50329.

In some embodiments, the pharmaceutical composition, e.g., pharmaceutical composition (e.g., composition of the total dose administered, e.g., once or twice daily) comprises about 12.8×10¹¹ to about 16×10¹¹ total cells of Prevotella histicola, e.g., of Prevotella Strain B 50329.

In some embodiments, the pharmaceutical composition is prepared as a solid dosage form. In certain embodiments, provided herein are solid dosage forms comprising the Prevotella histicola bacteria. In some embodiments, the solid dosage form comprises an enteric coating. In some embodiments, the solid dosage form is a tablet, e.g., an enteric coated tablet. In some embodiments, each tablet comprises about 3.2 x 10¹¹ total cells of the Prevotella histicola bacteria. In some embodiments, the solid dosage form is a capsule, e.g., an enteric coated capsule. In some embodiments, each capsule comprises about 3.2×10¹¹ total cells of the Prevotella histicola bacteria.

In some embodiments, 1 solid dosage form (e.g., tablet or capsule) is administered (e.g., is for administration) per day, wherein the solid dosage form comprises a dose of bacteria of about 3.2×10¹¹ total cells. In some embodiments, 2 solid dosage forms (e.g., tablets or capsules) are administered (e.g., are for administration) per day, wherein the solid dosage form comprises a dose of bacteria of about 3.2×10¹¹ total cells. In some embodiments, 3 solid dosage forms (e.g., tablets or capsules) are administered (e.g., are for administration) per day, wherein the solid dosage form comprises a dose of bacteria of about 3.2×10¹¹ total cells. In some embodiments, 4 solid dosage forms (e.g., tablets or capsules) are administered (e.g., are for administration) per a day, wherein the solid dosage form comprises a dose of bacteria of about 3.2×10¹¹ total cells. In some embodiments, 5 solid dosage forms (e.g., tablets or capsules) are administered (e.g., are for administration) per a day, wherein the solid dosage form comprises a dose of bacteria of about 3.2×10¹¹ total cells.

In some embodiments, the pharmaceutical composition, e.g., pharmaceutical composition is a powder. The powder can be resuspended (e.g., in a liquid such as a solution, buffer, water or other beverage or a food), e.g., for administration to a subject.

In some embodiments, a dose of Prevotella histicola bacteria of about 9.6×10¹¹ total cells are administered (e.g., are for administration) per day.

In some embodiments, a dose of Prevotella histicola bacteria of about 12.8×10¹¹ total cells are administered (e.g., are for administration) per day.

In some embodiments, a dose of Prevotella histicola bacteria of about 16×10¹¹ total cells are administered (e.g., are for administration) per day.

In some embodiments, the solid dosage form is a tablet. In some embodiments, the tablet is an enteric coated tablet. In some embodiments, the enteric coated tablet is from 5 mm to 18 mm in diameter (size refers to size prior to application of enteric coat). In some embodiments, the tablet comprises about 3.2×10¹¹ total cells of the Prevotella bacteria. In some embodiments, the Prevotella bacteria in the tablet are lyophilized.

In some embodiments, the solid dosage form is a capsule. In some embodiments, the capsule is an enteric coated capsule. In some embodiments, the enteric coated capsule is a size 00, size 0, size 1, size 2, size 3, size 4, or size 5 capsule. In some embodiments, the capsule is a size 0 capsule. In some embodiments, the capsule comprises about 3.2×10¹¹ total cells of the Prevotella bacteria. In some embodiments, the Prevotella bacteria in the capsule are lyophilized.

In certain embodiments, provided herein are solid dosage forms comprising the Prevotella bacteria. In some embodiments, the solid dosage form is a tablet, e.g., an enteric coated tablet. In some embodiments, the solid dosage form is a capsule, e.g., an enteric coated capsule. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P or Eudragit L30-D55).

In some embodiments, each tablet comprises about 3.2×10¹¹ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 3.2×10¹¹ total cells) is administered, e.g., once or twice daily to a subject. In some embodiments, 2 tablets (e.g., each comprising about 3.2×10¹¹ total cells) are administered, e.g., once or twice daily to a subject. In some embodiments, 3 tablets (e.g., each comprising about 3.2×10¹¹ total cells) are administered, e.g., once or twice daily to a subject. In some embodiments, 4 tablets (e.g., each comprising about 3.2 x 10¹¹ total cells) are administered, e.g., once or twice daily to a subject. In some embodiments, 6 tablets (e.g., each comprising about 3.2×10¹¹ total cells) are administered, e.g., once or twice daily to a subject. In some embodiments, 8 tablets (e.g., each comprising about 3.2×10¹¹ total cells) are administered, e.g., once or twice daily to a subject. In some embodiments, 10 tablets (e.g., each comprising about 3.2×10¹¹ total cells) are administered, e.g., once or twice daily to a subject. In some embodiments, the Prevotella bacteria in the tablet are lyophilized (e.g., in a powder). In some embodiments, the Prevotella bacteria in the tablet are lyophilized in a powder, and the powder further comprises mannitol, magnesium stearate, and/or colloidal silicon dioxide.

In some embodiments, each capsule comprises about 3.2×10¹¹ total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 3.2×10¹¹ total cells) is administered, e.g., once or twice daily to a subject. In some embodiments, 2 capsules (e.g., each comprising about 3.2×10¹¹ total cells) are administered, e.g., once or twice daily to a subject. In some embodiments, 3 capsules (e.g., each comprising about 3.2×10¹¹ total cells) are administered, e.g., once or twice daily to a subject. In some embodiments, 4 capsules (e.g., each comprising about 3.2×10¹¹ total cells) are administered, e.g., once or twice daily to a subject. In some embodiments, 6 capsules (e.g., each comprising about 3.2×10¹¹ total cells) are administered, e.g., once or twice daily to a subject. In some embodiments, 8 capsules (e.g., each comprising about 3.2×10¹¹ total cells) are administered, e.g., once or twice daily to a subject. In some embodiments, 10 capsules (e.g., each comprising about 3.2×10¹¹ total cells) are administered, e.g., once or twice daily to a subject. In some embodiments, the Prevotella bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the pharmaceutical composition comprises at least about 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰ 3×10¹⁰ 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 1.5×10¹² total cells of Prevotella histicola.

In some embodiments, the pharmaceutical composition comprises at most about 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹5×10¹¹, 6×10¹¹, 7×10¹¹, 8×10¹¹, 9×10¹¹,1×10¹², 1.5 ×10¹² total cells of Prevotella histicola.

In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are of the Prevotella strain. 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are of the Prevotella strain. In some embodiments, at least 99% of the bacteria in the composition are of the Prevotella strain. In some embodiments, the bacteria in the composition are essentially (e.g., about 100%) of the Prevotella strain.

In some embodiments, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the protein in the pharmaceutical composition is Prevotella strain bacteria protein.

In some embodiments, the pharmaceutical composition is formulated as a capsule or a tablet or a mini-tablet. In some embodiments, the pharmaceutical composition comprises an enteric coating or micro encapsulation. In some embodiments, the capsule is an enteric coated capsule. In some embodiments, the tablet is an enteric coated tablet. In some embodiments, the mini-tablet is an enteric coated mini-tablet.

In some embodiments, to quantify the numbers of Prevotella histicola bacteria present in a bacterial sample, electron microscopy (e.g., EM of ultrathin frozen sections) can be used to visualize the bacteria and count their relative numbers. Alternatively, combinations of nanoparticle tracking analysis (NTA), Coulter counting, and dynamic light scattering (DLS) or a combination of these techniques can be used. NTA and the Coulter counter count particles and show their sizes. DLS gives the size distribution of particles, but not the concentration. Bacteria frequently have diameters of 1-2 um. The full range is 0.2-20 um. Combined results from Coulter counting and NTA can reveal the numbers of bacteria in a given sample. Coulter counting reveals the numbers of particles with diameters of 0.7-10 um. NTA reveals the numbers of particles with diameters of 50-1400 nm. For most bacterial samples, the Coulter counter alone can reveal the number of bacteria in a sample. In some embodiments, total cells (total cell count) is determined by Coulter counting. In some embodiments, the Prevotella bacteria is quantified based on total cells, e.g., total cell count (TCC) (e.g., determined by Coulter counter).

In certain aspects, provided are pharmaceutical compositions for administration subjects. In some embodiments, the pharmaceutical compositions are combined with additional active and/or inactive materials in order to produce a final product, which may be in single dosage unit or in a multi-dose format. In some embodiments, the pharmaceutical composition is combined with an adjuvant such as an immuno-adjuvant (e.g., STING agonists, TLR agonists, NOD agonists).

In some embodiments the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments the composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof

In some embodiments the composition comprises at least one supplemental vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments the excipient is a buffering agent. Non-limiting examples of suitable buffering agents include sodium citrate, magnesium carbonate, magnesium bicarbonate, calcium carbonate, and calcium bicarbonate.

In some embodiments the excipient comprises a preservative. Non-limiting examples of suitable preservatives include antioxidants, such as alpha-tocopherol and ascorbate, and antimicrobials, such as parabens, chlorobutanol, and phenol.

In some embodiments the composition comprises a binder as an excipient. Non-limiting examples of suitable binders include starches, pregelatinized starches, gelatin, polyvinylpyrolidone, cellulose, methylcellulose, sodium carboxymethylce llulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments the composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Solid Dosage Form Composition

In certain embodiments, provided herein are solid dosage forms (solid dose forms) comprising a Prevotella strain and a pharmaceutically acceptable carrier.

In some embodiments, the pharmaceutical composition comprising Prevotella histicola bacteria is prepared as a powder (e.g., for resuspension or for use in a solid dosage form (such as a capsule)) or as a solid dosage form, such as a tablet, a mini-tablet, or a capsule; or a combination of these forms (e.g., mini-tablets comprised in a capsule)). The powder can comprise lyophilized bacteria. In some embodiments, the powder further comprises mannitol, magnesium stearate, and/or colloidal silicon dioxide. In some embodiments, the Prevotella histicola bacteria are gamma irradiated.

In some embodiments, the solid dosage forms comprise whole Prevotella histicola bacteria (e.g., live bacteria, killed bacteria, attenuated bacteria).

In some embodiments, the pharmaceutical compositions comprise live Prevotella histicola bacteria.

In some embodiments, the solid dosage forms comprise viable Prevotella histicola bacteria.

In certain embodiments, the solid dosage forms comprise non-viable Prevotella histicola bacteria.

In some embodiments, the solid dosage forms comprise only one strain of bacteria, e.g., Prevotella histicola , e.g., Prevotella Strain B 50329.

The solid dosage form (also referred to as solid dose form herein) can comprise one or more excipients, e.g., pharmaceutically acceptable excipients. The Prevotella histicola bacteria in the solid dosage form can be isolated Prevotella histicola bacteria. Optionally, the Prevotella histicola bacteria in the solid dosage form can be lyophilized. Optionally, the Prevotella histicola bacteria in the solid dosage form are non-viable. Optionally, the Prevotella histicola bacteria in the solid dosage form are gamma irradiated. The solid dosage form can comprise a tablet. The solid dosage form can comprise a capsule. The solid dosage form can comprise a tablet, a mini-tablet, a capsule, or a powder; or a combination of these forms (e.g., mini-tablets comprised in a capsule).

The Prevotella histicola bacteria in the solid dosage form can be in a powder (e.g., the powder comprises lyophilized Prevotella histicola bacteria). In some embodiments, the powder further comprises mannitol, magnesium stearate, and/or colloidal silicon dioxide. In some embodiments, the powder further comprises mannitol, magnesium stearate, and colloidal silicon dioxide. Optionally, the Prevotella histicola bacteria in the powder can be lyophilized. Optionally, the Prevotella histicola bacteria in the powder are live. Optionally, the Prevotella histicola bacteria in the powder are gamma irradiated.

In some embodiments, the lyophilized Prevotella histicola bacteria (e.g., powder) is resuspended (e.g., in a liquid such as a solution, buffer, water or other beverage or a food), e.g., for administration to a subject.

In certain embodiments, the pharmaceutical composition (e.g., pharmaceutical composition) provided herein is prepared as a solid dosage form comprising Prevotella histicola bacteria and a pharmaceutically acceptable carrier.

In certain embodiments, the pharmaceutical composition (e.g., pharmaceutical composition) provided herein is prepared as a solid dosage form comprising Prevotella histicola bacteria and a pharmaceutically acceptable carrier. The solid dosage form can comprise a tablet, a mini-tablet, a capsule, a pill, or a powder; or a combination of these forms (e.g., mini-tablets comprised in a capsule).

In some embodiments, the solid dosage form described herein can be a capsule, e.g., an enteric coated capsule. In some embodiments, the capsule is enteric coated, e.g., for duodenal release at pH 5.5. The capsule can be, e.g., a size 00, size 0, size 1, size 2, size 3, size 4, or size 5 capsule. In some embodiments, the capsule is a size 0 capsule. In some embodiments, the capsule comprises freeze-dried powder that comprises the Prevotella Strain.

In some embodiments, the solid dosage form described herein can be, e.g., a tablet or a mini-tablet. In some embodiments, a plurality of mini-tablets can be in (e.g., loaded into) a capsule.

In some embodiments, the solid dosage form comprises a tablet (>4 mm) (e.g., 5 mm-17 mm). In some embodiments, the tablet is enteric coated, e.g., for duodenal release at pH 5.5. For example, the tablet is a 5 mm, 6 mm, 7 mm, 8 mm, 9 mm, 10 mm, 11 mm, 12 mm, 13 mm, 14 mm, 15 mm, 16 mm, 17 mm or 18 mm tablet.

In some embodiments, the solid dosage form comprises a mini-tablet. In some embodiments, the mini-tablet is enteric coated, e.g., for duodenal release at pH 5.5. The mini-tablet can be in the size range of 1 mm-4 mm range. E.g., the mini-tablet can be a 1 mm mini-tablet, 1.5 mm mini-tablet, 2 mm mini-tablet, 3 mm mini-tablet, or 4 mm mini-tablet.

As used herein, the size of the tablet, mini-tablet or capsule refers to the size of the tablet, mini-tablet or capsule prior to application of an enteric coating.

In some embodiments, the solid dosage form comprises a mini-tablet. The mini-tablet can be in the size range of 1 mm-4 mm range. E.g., the mini-tablet can be a 1 mm mini-tablet, 1.5 mm mini-tablet, 2 mm mini-tablet, 3 mm mini-tablet, or 4 mm mini-tablet. The size refers to the diameter of the mini-tablet, as is known in the art. As used herein, the size of the mini-tablet refers to the size of the mini-tablet prior to application of an enteric coating.

The mini-tablets can be in a capsule. The capsule can be a size 00, size 0, size 1, size 2, size 3, size 4, or size 5 capsule. The capsule that contains the mini-tablets can comprise a single layer coating, e.g., a non-enteric coating such as gelatin or HPMC. The mini-tablets can be inside a capsule: the number of mini-tablets inside a capsule will depend on the size of the capsule and the size of the mini-tablets. As an example, a size 0 capsule can contain 31-35 (an average of 33) mini-tablets that are 3 mm mini-tablets.

The solid dosage form (e.g., tablet, mini-tablet, or capsule) described herein can be enterically coated. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

The solid dose form can comprise a coating. The solid dose form can comprise a single layer coating, e.g., enteric coating, e.g., a Eudragit-based coating, e.g., EUDRAGIT L30 D-55, triethylcitrate, and talc. The solid dose form can comprise two layers of coating. For example, an inner coating can comprise, e.g., EUDRAGIT L30 D-55, triethylcitrate, talc, citric acid anhydrous, and sodium hydroxide, and an outer coating can comprise, e.g., EUDRAGIT L30 D-55, triethylcitrate, and talc. EUDRAGIT is the brand name for a diverse range of polymethacrylate-based copolymers. It includes anionic, cationic, and neutral copolymers based on methacrylic acid and methacrylic/acrylic esters or their derivatives. Eudragits are amorphous polymers having glass transition temperatures between 9 to >150° C. Eudragits are non-biodegradable, nonabsorbable, and nontoxic. Anionic Eudragit L dissolves at pH>6 and is used for enteric coating, while Eudragit S, soluble at pH>7 is used for colon targeting. Eudragit RL and RS, having quaternary ammonium groups, are water insoluble, but swellable/permeable polymers which are suitable for the sustained release film coating applications. Cationic Eudragit E, insoluble at pH≥5, can prevent drug release in saliva.

The solid dose form (e.g., a capsule) can comprise a single layer coating, e.g., a non-enteric coating such as gelatin or HPMC. For example, enteric coated mini-tablets can be in a gelatin or HPMC capsule.

A pharmaceutical composition comprising Prevotella histicola bacteria can be formulated as a suspension, e.g., for oral administration or for injection. Administration by injection includes intravenous (IV), intramuscular (IM), and subcutaneous (SC) administration. For a suspension, Prevotella histicola bacteria can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The Prevotella bacteria in the suspension can be isolated Prevotella histicola bacteria. Optionally, the Prevotella histicola bacteria in the suspension can be lyophilized. Optionally, the Prevotella histicola bacteria in the solid dose form are live. Optionally, the Prevotella histicola bacteria in the suspension can be gamma irradiated.

### Dosage

For oral administration to a human subject, the dose of Prevotella histicola bacteria can be, e.g., about 2×10⁶- about 2×10¹⁶ particles. The dose can be, e.g., about 1×10⁷- about 1×10¹⁵, about 1×10⁸- about 1×10¹⁴, about 1×10⁹- about 1×10¹³, about 1×10¹⁰- about 1×10¹⁴, or about 1×10⁸- about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 2×10¹⁴ particles. The dose can be, e.g., about 2×10¹² particles. The dose can be, e.g., about 2×10¹⁰ particles. The dose can be, e.g., about 1×10¹⁰ particles. Particle count can be determined, e.g., by NTA.

For oral administration to a human subject, the dose of Prevotella histicola bacteria can be, e.g., based on total protein. The dose can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 10 mg total protein. Total protein can be determined, e.g., by Bradford assay or by the BCA assay.

For administration by injection (e.g., intravenous administration) to a human subject, the dose of Prevotella histicola bacteria can be, e.g., about 1×10⁶- about 1×10¹⁶ particles. The dose can be, e.g., about 1×10⁷- about 1×10¹⁵, about 1×10⁸- about 1×10¹⁴, about 1×10⁹- about 1×10¹³, about 1×10¹⁰- about 1×10¹⁴, or about 1×10⁸- about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 1×10¹⁵ particles. The dose can be, e.g., about 2×10¹⁴ particles. The dose can be, e.g., about 2×10¹³ particles. Particle count can be determined, e.g., by NTA.

For administration by injection (e.g., intravenous administration), the dose of Prevotella histicola bacteria can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 700 mg total protein. The dose can be, e.g., about 350 mg total protein. The dose can be, e.g., about 175 mg total protein. Total protein can be determined, e.g., by Bradford assay or by the BCA assay.

In certain embodiments, the pharmaceutical composition (e.g., composition of the total dose administered, e.g., once or twice daily) comprises at least 1×10¹⁰ total cells (e.g., at least 1×10¹⁰ total cells, at least 2×10¹⁰ total cells, at least 3×10¹⁰ total cells, at least 4×10¹⁰ total cells, at least 5×10¹⁰ total cells, at least 6×10¹⁰ total cells, at least 7×10¹⁰ total cells, at least 8×10¹⁰ total cells, at least 9×10¹⁰ total cells, at least 1×10¹¹ total cells of the Prevotella histicola bacteria. In some embodiments, the pharmaceutical composition comprises no more than 20×10¹¹ total cells (e.g., no more than 1×10¹⁰ total cells, no more than 2×10¹⁰ total cells, no more than 3×10¹⁰ total cells, no more than 4×10¹⁰ total cells, no more than 5×10¹⁰ total cells, no more than 6×10¹⁰ total cells, no more than 7×10¹⁰ total cells, no more than 8×10¹⁰ total cells, no more than 9×10¹⁰ total cells, no more than 1×10¹¹ total cells, no more than 2×10¹¹ total cells, no more than 3×10¹¹ total cells, no more than 4×10¹¹ total cells, no more than 5×10¹¹ total cells, no more than 6×10¹¹ total cells, no more than 7×10¹¹ total cells, no more than 8×10¹¹ total cells, no more than 9×10¹¹ total cells, no more than 10×10¹¹ total cells, no more than 11×10¹¹ total cells, no more than 12×10¹¹ total cells, no more than 13×10¹¹ total cells, no more than 14×10¹¹ total cells, no more than 15×10¹¹ total cells, no more than 16×10¹¹ total cells, no more than 17×10¹¹ total cells, no more than 18×10¹¹ total cells, no more than 19×10¹¹ total cells,) of the Prevotella histicola bacteria. In some embodiments, the pharmaceutical composition comprises about 6×10⁹ total cells of the Prevotella histicola bacteria. In some embodiments, the pharmaceutical composition comprises about 1.6×10¹⁰ total cells of the Prevotella histicola bacteria. In some embodiments, the pharmaceutical composition comprises about 8×10¹⁰ total cells of the Prevotella histicola bacteria. In some embodiments, the pharmaceutical composition comprises about 1.6×10¹¹ total cells the Prevotella histicola bacteria. In some embodiments, the pharmaceutical composition comprises about 3.2×10¹¹ total cells the Prevotella histicola bacteria. In some embodiments, the pharmaceutical composition comprises about 8×10¹¹ total cells of the Prevotella histicola bacteria. In some embodiments, the pharmaceutical composition comprises about 9.6×10¹¹ total cells of the Prevotella histicola bacteria. In some embodiments, the pharmaceutical composition comprises about 12.8×10¹¹ total cells of the Prevotella histicola bacteria. In some embodiments, the pharmaceutical composition comprises about 16×10¹¹ total cells of the Prevotella histicola bacteria. In some embodiments, the pharmaceutical composition comprises about 1.6×10¹⁰ to about 8×10¹¹ total cells of the Prevotella histicola bacteria. In some embodiments, the pharmaceutical composition comprises about 1.6×10¹⁰ to about 1.6×10¹¹total cells of the Prevotella histicola bacteria. In some embodiments, the pharmaceutical composition comprises about 1.6×10¹⁰ to about 16×10¹¹total cells of the Prevotella histicola bacteria. In some embodiments, the pharmaceutical composition comprises about 8×10¹⁰ to about 8×10¹¹ total cells of the Prevotella histicola bacteria. In some embodiments, the pharmaceutical composition comprises about 9.6×10¹¹to about 16×10¹¹ total cells of the Prevotella histicola bacteria. In some embodiments, the pharmaceutical composition comprises about 9.6×10¹¹to about 12.8×10¹¹ total cells of the Prevotella histicola bacteria. In some embodiments, the pharmaceutical composition comprises about 12.8×10¹¹to about 16×10¹¹ total cells of the Prevotella histicola bacteria.

In some embodiments, the pharmaceutical composition (e.g., composition of the total dose administered, e.g., once or twice daily) comprises about 8×10¹⁰ to about 8×10¹¹ total cells of Prevotella histicola , e.g., of Prevotella Strain B 50329.

In some embodiments, the pharmaceutical composition (e.g., composition of the total dose administered, e.g., once or twice daily) comprises about 1.6×10¹⁰ to about 1.6×10¹¹ total cells of Prevotella histicola , e.g., of Prevotella Strain B 50329.

In some embodiments, the pharmaceutical composition (e.g., composition of the total dose administered, e.g., once or twice daily) comprises about 1.6×10¹⁰ to about 16×10¹¹ total cells of Prevotella histicola , e.g., of Prevotella Strain B 50329.

In some embodiments, the pharmaceutical composition (e.g., composition of the total dose administered, e.g., once or twice daily) comprises about 8×10¹⁰ total cells of Prevotella histicola , e.g., of Prevotella Strain B 50329.

In some embodiments, the pharmaceutical composition (e.g., composition of the total dose administered, e.g., once or twice daily) comprises about 1.6×10¹¹total cells of Prevotella histicola , e.g., of Prevotella Strain B 50329.

In some embodiments, the pharmaceutical composition (e.g., composition of the total dose administered, e.g., once or twice daily) comprises about 3.2×10¹¹total cells of Prevotella histicola , e.g., of Prevotella Strain B 50329.

In some embodiments, the pharmaceutical composition (e.g., composition of the total dose administered, e.g., once or twice daily) comprises about 8×10¹¹total cells of Prevotella histicola , e.g., of Prevotella Strain B 50329.

In some embodiments, the pharmaceutical composition (e.g., composition of the total dose administered, e.g., once or twice daily) comprises about 9.6×10¹¹total cells of Prevotella histicola , e.g., of Prevotella Strain B 50329.

In some embodiments, the pharmaceutical composition (e.g., composition of the total dose administered, e.g., once or twice daily) comprises about 12.8×10¹¹total cells of Prevotella histicola , e.g., of Prevotella Strain B 50329.

In some embodiments, the pharmaceutical composition (e.g., composition of the total dose administered, e.g., once or twice daily) comprises about 16×10¹¹total cells of Prevotella histicola , e.g., of Prevotella Strain B 50329.

In some embodiments, the pharmaceutical composition (e.g., composition of the total dose administered, e.g., once or twice daily) comprises about 9.6×10¹¹to about 16×10¹¹ total cells of Prevotella histicola , e.g., of Prevotella Strain B 50329.

In some embodiments, the pharmaceutical composition (e.g., composition of the total dose administered, e.g., once or twice daily) comprises about 9.6×10¹¹to about 12.8×10¹¹ total cells of Prevotella histicola , e.g., of Prevotella Strain B 50329.

In some embodiments, the pharmaceutical composition (e.g., composition of the total dose administered, e.g., once or twice daily) comprises about 12.8×10¹¹to about 16 x 10¹¹ total cells of Prevotella histicola , e.g., of Prevotella Strain B 50329.

In some embodiments, the Prevotella histicola bacteria may be quantified based on total cells, e.g., total cell count (TCC) (e.g., determined by Coulter counter).

In certain embodiments, provided herein are solid dosage forms comprising the Prevotella histicola bacteria. In some embodiments, the solid dosage form comprises an enteric coating.

In some embodiments, the solid dosage form is a capsule, e.g., an enteric coated capsule. In some embodiments, each capsule comprises about 8×10¹⁰ total cells of the Prevotella histicola bacteria. In some embodiments, 1, 2, 3, 4, 5, 6, 7, 8, 9, or 10 capsules are administered, e.g., once or twice daily to a subject. In some embodiments, 1 capsule (e.g., comprising about 8×10¹⁰ total cells) is administered, e.g., once or twice daily to a subject. In some embodiments, 2 capsules (e.g., each comprising about 8×10¹⁰ total cells) are administered, e.g., once or twice daily to a subject. In some embodiments, 4 capsules (e.g., each comprising about 8×10¹⁰ total cells) are administered, e.g., once or twice daily to a subject. In some embodiments, 5 capsules (e.g., each comprising about 8 x 10¹⁰ total cells) are administered, e.g., once or twice daily to a subject. In some embodiments, 10 capsules (e.g., each comprising about 8×10¹⁰ total cells) are administered, e.g., once or twice daily to a subject. In some embodiments, each capsule comprises about 1.6×10¹¹ total cells of the Prevotella histicola bacteria. In some embodiments, 1, 2, 3, 4, 5, 6, 7, 8, 9, or 10 capsules are administered, e.g., once or twice daily to a subject. In some embodiments, 1 capsule (e.g., comprising about 1.6×10¹¹ total cells) is administered, e.g., once or twice daily to a subject. In some embodiments, 2 capsules (e.g., each comprising about 1.6×10¹¹ total cells) are administered, e.g., once or twice daily to a subject. In some embodiments, 4 capsules (e.g., each comprising about 1.6×10¹¹ total cells) are administered, e.g., once or twice daily to a subject. In some embodiments, 5 capsules (e.g., each comprising about 1.6×10¹¹ total cells) are administered, e.g., once or twice daily to a subject. In some embodiments, 10 capsules (e.g., each comprising about 1.6×10¹¹ total cells) are administered, e.g., once or twice daily to a subject. In some embodiments, each capsule comprises about 3.2×10¹¹ total cells of the Prevotella histicola bacteria. In some embodiments, 1, 2, 3, 4, 5, 6, 7, 8, 9, or 10 capsules are administered, e.g., once or twice daily to a subject. In some embodiments, 1 capsule (e.g., comprising about 3.2×10¹¹ total cells) is administered, e.g., once or twice daily to a subject. In some embodiments, 2 capsules (e.g., each comprising about 3.2×10¹¹ total cells) are administered, e.g., once or twice daily to a subject. In some embodiments, 4 capsules (e.g., each comprising about 3.2×10¹¹ total cells) are administered, e.g., once or twice daily to a subject. In some embodiments, 5 capsules (e.g., each comprising about 3.2×10¹¹ total cells) are administered, e.g., once or twice daily to a subject. In some embodiments, 10 capsules (e.g., each comprising about 3.2×10¹¹ total cells) are administered, e.g., once or twice daily to a subject. In some embodiments, the Prevotella histicola bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a capsule. In some embodiments, the capsule is an enteric coated capsule. In some embodiments, the capsule comprises about 8×10¹⁰ total cells of the Prevotella histicola bacteria (e.g., total dose of a capsule or plurality of capsules). In some embodiments, the capsule comprises about 1.6×10¹¹ total cells of the Prevotella histicola bacteria (e.g., total dose of a capsule or plurality of capsules). In some embodiments, the capsule comprises about 3.2×10¹¹ total cells of the Prevotella histicola bacteria (e.g., total dose of a capsule or plurality of capsules). In some embodiments, the capsule comprises about 8×10¹¹ total cells of the Prevotella histicola bacteria (e.g., total dose of a capsule or plurality of capsules). In some embodiments, the capsule comprises about 9.6×10¹¹ total cells of the Prevotella histicola bacteria (e.g., total dose of a capsule or plurality of capsules). In some embodiments, the capsule comprises about 12.8×10¹¹ total cells of the Prevotella histicola bacteria (e.g., total dose of a capsule or plurality of capsules). In some embodiments, the capsule comprises about 16×10¹¹ total cells of the Prevotella histicola bacteria (e.g., total dose of a capsule or plurality of capsules). In some embodiments, the Prevotella histicola bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a tablet. In some embodiments, the tablet is an enteric coated tablet. In some embodiments, the enteric coated tablet is from 5 mm to 18 mm in diameter. In some embodiments, the tablet comprises about 8×10¹⁰ total cells of the Prevotella histicola bacteria (e.g., total dose of a tablet or plurality of tablets). In some embodiments, the tablet comprises about 1.6 x 10¹¹ total cells of the Prevotella histicola bacteria (e.g., total dose of a tablet or plurality of tablets). In some embodiments, the tablet comprises about 3.2×10¹¹ total cells of the Prevotella histicola bacteria (e.g., total dose of a tablet or plurality of tablets). In some embodiments, the tablet comprises about 8×10¹¹ total cells of the Prevotella histicola bacteria (e.g., total dose of a tablet or plurality of tablets). In some embodiments, the tablet comprises about 9.6×10¹¹ total cells of the Prevotella histicola bacteria (e.g., total dose of a tablet or plurality of tablets). In some embodiments, the tablet comprises about 12.8×10¹¹ total cells of the Prevotella histicola bacteria (e.g., total dose of a tablet or plurality of tablets). In some embodiments, the tablet comprises about 16×10¹¹ total cells of the Prevotella histicola bacteria (e.g., total dose of a tablet or plurality of tablets). In some embodiments, the Prevotella histicola bacteria in the tablet are lyophilized (e.g., in a powder).

In some embodiments, the solid dosage form comprises a mini-tablet. In some embodiments, the mini-tablet is enteric coated. In some embodiments, the mini-tablet is from 1 mm to 4 mm in diameter. In some embodiments, the mini-tablet (e.g., enteric coated mini-tablet) is a 1 mm mini-tablet, 1.5 mm mini-tablet, 2 mm mini-tablet, 3 mm mini-tablet, or 4 mm mini-tablet. In some embodiments, the solid dosage form comprises mini-tablets that comprise about 8×10¹⁰ total cells of the Prevotella histicola bacteria (e.g., total dose of a plurality of mini-tablets). In some embodiments, the solid dosage form comprises mini-tablets that comprise about 1.6×10¹¹ total cells of the Prevotella histicola bacteria (e.g., total dose of a plurality of mini-tablets). In some embodiments, the solid dosage form comprises mini-tablets that comprise about 3.2×10¹¹ total cells of the Prevotella histicola bacteria (e.g., total dose of a plurality of mini-tablets). In some embodiments, the solid dosage form comprises mini-tablets that comprise about 8×10¹¹ total cells of the Prevotella histicola bacteria (e.g., total dose of a plurality of mini-tablets). In some embodiments, the solid dosage form comprises mini-tablets that comprise about 9.6×10¹¹ total cells of the Prevotella histicola bacteria (e.g., total dose of a plurality of mini-tablets). In some embodiments, the solid dosage form comprises mini-tablets that comprise about 12.8×10¹¹ total cells of the Prevotella histicola bacteria (e.g., total dose of a plurality of mini-tablets). In some embodiments, the solid dosage form comprises mini-tablets that comprise about 16×10¹¹ total cells of the Prevotella histicola bacteria (e.g., total dose of a plurality of mini-tablets). In some embodiments, the Prevotella histicola bacteria in the mini-tablets are lyophilized (e.g., in a powder). In some embodiments, the mini-tablets (e.g., enteric coated mini-tablets) are contained in a capsule. In some embodiments, the capsule is a size 00, size 0, size 1, size 2, size 3, size 4, or size 5 capsule. In some embodiments, the capsule comprises a non-enteric coating (e.g., gelatin or HMC) (e.g., is coated with a non-enteric coating). In some embodiments, the capsule comprises a non-enteric coating. In some embodiments, the capsule comprises gelatin. In some embodiments, the capsule comprises HPMC. In some embodiments, the mini-tablets (e.g., enteric coated mini-tablets) that comprise about 8×10¹⁰ total cells of the Prevotella histicola bacteria are contained in a capsule(s), wherein optionally the capsule comprises gelatin or HPMC. In some embodiments, the mini-tablets (e.g., enteric coated mini-tablets) that comprise about 1.6×10¹¹ total cells of the Prevotella histicola bacteria are contained in a capsule(s), wherein optionally the capsule comprises gelatin or HPMC. In some embodiments, the mini-tablets (e.g., enteric coated mini-tablets) that comprise about 8×10¹¹ total cells of the Prevotella histicola bacteria are contained in a capsule(s), wherein optionally the capsule comprises gelatin or HPMC. In some embodiments, the mini-tablets (e.g., enteric coated mini-tablets) that comprise about 9.6×10¹¹ total cells of the Prevotella histicola bacteria are contained in a capsule(s), wherein optionally the capsule comprises gelatin or HPMC. In some embodiments, the mini-tablets (e.g., enteric coated mini-tablets) that comprise about 12.88×10¹¹ total cells of the Prevotella histicola bacteria are contained in a capsule(s), wherein optionally the capsule comprises gelatin or HPMC. In some embodiments, the mini-tablets (e.g., enteric coated mini-tablets) that comprise about 16×10¹¹ total cells of the Prevotella histicola bacteria are contained in a capsule(s), wherein optionally the capsule comprises gelatin or HPMC.

In certain embodiments, provided herein are solid dosage forms comprising the Prevotella histicola bacteria. In some embodiments, the solid dosage form comprises an enteric coating. In some embodiments, the solid dosage form is a tablet, e.g., an enteric coated tablet. In some embodiments, each tablet comprises about 3.2×10¹¹ total cells of the Prevotella histicola bacteria. In some embodiments, the solid dosage form is a mini-tablet, e.g., an enteric coated mini-tablet. In some embodiments, the total dose of a plurality of mini-tablets (e.g., mini-tablets contained in a capsule) comprises about 3.2×10¹¹ total cells of the Prevotella histicola bacteria. In some embodiments, the solid dosage form is a capsule, e.g., an enteric coated capsule. In some embodiments, each capsule comprises about 3.2×10¹¹ total cells of the Prevotella histicola bacteria.

In some embodiments, the mini-tablets (e.g., enteric coated mini-tablets) are contained in a capsule. In some embodiments, the capsule is a size 00, size 0, size 1, size 2, size 3, size 4, or size 5 capsule. In some embodiments, the capsule comprises a non-enteric coating (e.g., gelatin or HPMC) (e.g., is coated with a non-enteric coating). In some embodiments, the capsule comprises a non-enteric coating. In some embodiments, the capsule comprises gelatin. In some embodiments, the capsule comprises HPMC. In some embodiments, the total dose of a plurality of mini-tablets (e.g., mini-tablets contained in a capsule (e.g., enteric coated mini-tablets in a non-enteric coated capsule)) comprises about 3.2×10¹¹ total cells of the Prevotella histicola bacteria.

In some embodiments, 1 solid dosage form (e.g., tablet or capsule) is administered (e.g., is for administration) per day, wherein the solid dosage form comprises a dose of bacteria of about 3.2×10¹¹ total cells. In some embodiments, 2 solid dosage forms (e.g., tablets or capsules) are administered (e.g., are for administration) per day, wherein the solid dosage form (e.g., each solid dosage form) comprises a dose of bacteria of about 3.2×10¹¹ total cells. In some embodiments, 3 solid dosage forms (e.g., tablets or capsules) are administered (e.g., are for administration) per day, wherein the solid dosage form comprises a dose of bacteria of about 3.2×10¹¹ total cells. In some embodiments, 4 solid dosage forms (e.g., tablets or capsules) are administered (e.g., are for administration) per a day, wherein the solid dosage form comprises a dose of bacteria of about 3.2×10¹¹ total cells. In some embodiments, 5 solid dosage forms (e.g., tablets or capsules) are administered (e.g., are for administration) per a day, wherein the solid dosage form comprises a dose of bacteria of about 3.2×10¹¹ total cells. In some embodiments, 6 solid dosage forms (e.g., tablets or capsules) are administered (e.g., are for administration) per a day, wherein the solid dosage form comprises a dose of bacteria of about 3.2×10¹¹ total cells. In some embodiments, 8 solid dosage forms (e.g., tablets or capsules) are administered (e.g., are for administration) per a day, wherein the solid dosage form comprises a dose of bacteria of about 3.2×10¹¹ total cells. In some embodiments, 10 solid dosage forms (e.g., tablets or capsules) are administered (e.g., are for administration) per a day, wherein the solid dosage form comprises a dose of bacteria of about 3.2×10¹¹ total cells.

The capsule can be, for example, a capsule (e.g., enteric coated capsule) comprising Prevotella histicola bacteria (e.g., a powder thereof). The capsule can be, for example, a capsule (non-enteric coated) that contains mini-tablets (e.g., enteric coated mini-tablets) comprising Prevotella histicola bacteria.

In some embodiments, a dose of Prevotella histicola bacteria of about 9.6×10¹¹ total cells are administered (e.g., are for administration) per day.

In some embodiments, a dose of Prevotella histicola bacteria of about 12.8×10¹¹ total cells are administered (e.g., are for administration) per day.

In some embodiments, a dose of Prevotella histicola bacteria of about 16×10¹¹ total cells are administered (e.g., are for administration) per day.

In some embodiments, the solid dosage form is a tablet. In some embodiments, the tablet is an enteric coated tablet. In some embodiments, the enteric coated tablet is from 5 mm to 18 mm in diameter. In some embodiments, the tablet comprises about 3.2×10¹¹ total cells of the Prevotella bacteria. In some embodiments, the Prevotella bacteria in the tablet are lyophilized.

In some embodiments, the solid dosage form is a capsule. In some embodiments, the capsule is an enteric coated capsule. In some embodiments, the enteric coated capsule is a size 00, size 0, size 1, size 2, size 3, size 4, or size 5 capsule. In some embodiments, the capsule is a size 0 capsule. In some embodiments, the capsule comprises about 3.2×10¹¹ total cells of the Prevotella bacteria. In some embodiments, the Prevotella bacteria in the capsule are lyophilized.

In certain embodiments, provided herein are solid dosage forms comprising the Prevotella bacteria. In some embodiments, the solid dosage form is a tablet, e.g., an enteric coated tablet. In some embodiments, the solid dosage form is a mini-tablet, e.g., an enteric coated mini-tablet. In some embodiments, the solid dosage form is a capsule, e.g., an enteric coated capsule. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P or Eudragit L30-D55).

In some embodiments, each tablet comprises about 3.2×10¹¹ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 3.2×10¹¹ total cells) is administered, e.g., once or twice daily to a subject. In some embodiments, 2 tablets (e.g., each comprising about 3.2×10¹¹ total cells) are administered, e.g., once or twice daily to a subject. In some embodiments, 3 tablets (e.g., each comprising about 3.2×10¹¹ total cells) are administered, e.g., once or twice daily to a subject. In some embodiments, 4 tablets (e.g., each comprising about 3.2 x 10¹¹ total cells) are administered, e.g., once or twice daily to a subject. In some embodiments, 6 tablets (e.g., each comprising about 3.2×10¹¹ total cells) are administered, e.g., once or twice daily to a subject. In some embodiments, 8 tablets (e.g., each comprising about 3.2×10¹¹ total cells) are administered, e.g., once or twice daily to a subject. In some embodiments, 10 tablets (e.g., each comprising about 3.2×10¹¹ total cells) are administered, e.g., once or twice daily to a subject. In some embodiments, the Prevotella bacteria in the tablet are lyophilized (e.g., in a powder). In some embodiments, the Prevotella bacteria in the tablet are lyophilized in a powder, and the powder further comprises mannitol, magnesium stearate, and/or colloidal silicon dioxide.

In some embodiments, each capsule comprises about 3.2×10¹¹ total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 3.2×10¹¹ total cells) is administered, e.g., once or twice daily to a subject. In some embodiments, 2 capsules (e.g., each comprising about 3.2×10¹¹ total cells) are administered, e.g., once or twice daily to a subject. In some embodiments, 3 capsules (e.g., each comprising about 3.2×10¹¹ total cells) are administered, e.g., once or twice daily to a subject. In some embodiments, 4 capsules (e.g., each comprising about 3.2×10¹¹ total cells) are administered, e.g., once or twice daily to a subject. In some embodiments, 6 capsules (e.g., each comprising about 3.2×10¹¹ total cells) are administered, e.g., once or twice daily to a subject. In some embodiments, 8 capsules (e.g., each comprising about 3.2×10¹¹ total cells) are administered, e.g., once or twice daily to a subject. In some embodiments, 10 capsules (e.g., each comprising about 3.2×10¹¹ total cells) are administered, e.g., once or twice daily to a subject. In some embodiments, the Prevotella bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the Prevotella histicola bacteria are quantified based on total cells, e.g., total cell count (TCC) (e.g., determined by Coulter counter).

### Gamma-Irradiation

Powders (e.g., of Prevotella histicola bacteria) can be gamma-irradiated at 17.5 kGy radiation unit at ambient temperature.

Frozen biomasses (e.g., of Prevotella histicola bacteria) can be gamma-irradiated at 25 kGy radiation unit in the presence of dry ice.

### Additional Therapy

In some embodiments, an additional therapy is administered to the subject. In some embodiments, the additional therapy comprises an antiviral medication. In some embodiments, the additional therapy comprises an antiviral medication such as ribavirin, neuraminidase inhibitor, protease inhibitor, recombinant interferons, antibodies, oseltamivir, zanamivir, peramivir or baloxavir marboxil. In some embodiments, the additional therapy comprises hydroxychloroquine and/or chloroquine. In some embodiments, the additional therapy comprises remdesivir. In some embodiments, the additional therapy comprises plasma from a subject who has recovered from infection by the same virus that is infecting the subject (e.g., plasma from a subject who has recovered from SARS-CoV-2 infection) (e.g., convalescent plasma therapy).

In some embodiments, the additional therapy comprises an anti-inflammatory agent such as an NSAID or an anti-inflammatory steroid. In some embodiments, the additional therapy comprises a corticosteroid such as dexamethasone, prednisone, methylprednisolone, or hydrocortisone. In some embodiments, the additional therapy comprises dexamethasone.

In some embodiments, the additional therapy comprises an antibody specific for IL-6 and/or the IL-6 receptor. In some embodiments, the additional therapy comprises tocilizumab (Actemra®). In some embodiments, the additional therapy comprises sarilumab (Kevzara®).

In some embodiments, the additional therapy can comprise an anti-viral therapy. For example, the anti-viral therapy can comprise a nucleotide analog, such as remdesivir, galidesivir or clevudine; a viral RNA polymerase inhibitor such as favipiravir or galidesivir; a protease inhibitor such as ritonavir, darunavir, or danoprevir; an inhibitor of viral membrane fusion such as umifenovir; and/or anti-SARS-CoV-2 plasma.

In some embodiments, the additional therapy can comprise an anti-inflammatory therapy. For example, the anti-inflammatory therapy can comprise a corticosteroid; sirolimus; anakinra; filamod; or an antibody. In some embodiments, the antibody can comprise a GMSF inhibitor, such as lenzilumab or gimsilumab; an anti-IL1 beta inhibitor such as canakinumab; an IL-6 inhibitor such as tocilizumab or siltuximab; an IL-6R inhibitor such as sarilumab; and/or a CCR5 antagonist such as leronlimab.

In some embodiments, the additional therapy can comprise a JAK inhibitor such as baricitinib, ruxolitinib, tofacitinib, and/or pacritinib. In some embodiments, the additional therapy can comprise baricitinib. In some embodiments, the additional therapy can comprise baricitinib in combination with remdesivir.

In some embodiments, the additional therapy can comprise a TLR7 agonist such as imiquimod or reisquimod.

In some embodiments, the additional therapy can comprise a cell based therapy. For example, the cell based therapy can comprise Remestemcel-L; bone marrow stem cell therapy, such as Multi Stem or Bm-Allo-MSC; mesenchymal stromal cells; and/or adiopose derived mesenchymal stem cells such as AstroStem.

In some embodiments, the additional therapy can comprise an ACE receptor inhibitor. In some embodiments, the additional therapy can comprise an angiotensin-converting enzyme (ACE) inhibitor. In some embodiments, the additional therapy can comprise an angiotensin-converting enzyme 2 (ACE2) inhibitor.

In some embodiments, the additional therapy can comprise a regulator of the Sigma 1 and/or Sigma 2 receptor.

In some embodiments, the additional therapy can comprise IFN-β1a (e.g., by inhalation). In some embodiments, the additional therapy can comprise SNG001 (IFN-β1a for Nebulisation).

In some embodiments, the additional therapy can comprise a monoclonal antibody treatment. In some embodiments, the additional therapy can comprise a monoclonal antibody treatment such as bamlanivimab, casirivimab, or imdevimab, or a combination thereof, e.g., a combination of casirivimab and imdevimab. In some embodiments, the additional therapy can comprise a monoclonal antibody treatment such as bamlanivimab or etesevimab, or a combination of bamlanivimab or etesevimab.

In some embodiments, the additional therapy can comprise budesonide, e.g., inhaled budesonide.

In some embodiments, the additional therapy can comprise an anticoagulation drug, such as heparin or enoxaparin (e.g., a low-dose thereof).

In some embodiments, the additional therapy can comprise vitamin D.

In some embodiments, the additional therapy can comprise plitidepsin (also referred to as dehydrodidemnin B) (e.g., marketed as Aplidin).

In some embodiments, the additional therapy can comprise ivermectin.

### Administration

In certain aspects, provided herein is a method of delivering a pharmaceutical composition described herein to a subject. In some embodiments, the subject is a mammal. In some embodiments, the subject is a human.

In some embodiments, the pharmaceutical composition is administered orally. In some embodiments, the administration to the subject for a single day followed by a washout period before the next dose. In some embodiments, the washout period is at least 12 hours, 24 hours, 36 hours, 48 hours, 50 hours, 60 hours, or 72 hours.

In some embodiments, the pharmaceutical composition is administered after the washout period once daily for 14 days, 15 days, 16 days, 17 days, 18 days, 19 days, 20 days, 21 days, 22 days, 23 days, 24 days, 25 days, 26 days, 27 days, or 28 days.

In some embodiments, the pharmaceutical composition is administered after the washout period twice daily for 14 days, 15 days, 16 days, 17 days, 18 days, 19 days, 20 days, 21 days, 22 days, 23 days, 24 days, 25 days, 26 days, 27 days, or 28 days.

In some embodiments, the pharmaceutical composition is administered for 14 days, 15 days, 16 days, 17 days, 18 days, 19 days, 20 days, 21 days, 22 days, 23 days, 24 days, 25 days, 26 days, 27 days, or 28 days. In some embodiments, the pharmaceutical composition is administered for 14 days. In some embodiments, the pharmaceutical composition is administered for 21 days.

In some embodiments, the pharmaceutical composition is administered twice daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In some embodiments, the pharmaceutical composition is administered twice daily for 14 days. In some embodiments, the pharmaceutical composition is administered twice daily for 21 days.

In some embodiments, the pharmaceutical composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In some embodiments, the pharmaceutical composition is administered once daily for 14 days. In some embodiments, the pharmaceutical composition is administered once daily for 21 days.

In some embodiments, the pharmaceutical composition is administered twice daily for three days and then once daily for the remainder of the treatment (e.g., until day 14).

In some embodiments, the pharmaceutical composition is formulated as a capsule (e.g., containing mini-tablets or powder) or a tablet. In some embodiments, the pharmaceutical composition comprises an enteric coating or micro encapsulation. In some embodiments, the capsule is an enteric coated capsule. In some embodiments, the capsule is an HPMC capsule, e.g., that is further enteric coated. In some embodiments, the capsule is a gelatin capsule, e.g., that is further enteric coated.

In some embodiments of the methods provided herein, the pharmaceutical composition is administered in conjunction with the administration of an additional therapeutic. In some embodiments, the pharmaceutical composition comprises Prevotella histicola bacteria co-formulated with the additional therapeutic. In some embodiments, the pharmaceutical composition is co-administered with the additional therapeutic. In some embodiments, the additional therapeutic is administered to the subject before administration of the pharmaceutical composition (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic is administered to the subject after administration of the pharmaceutical composition (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the pharmaceutical composition and the additional therapeutic. In some embodiments different modes of delivery are used to administer the pharmaceutical composition and the additional therapeutic. For example, in some embodiments the pharmaceutical composition is administered orally while the additional therapeutic is administered via injection (e.g., an intravenous, and/or intramuscular injection).

In some embodiments, the pharmaceutical composition is administered orally. In some embodiments, the administration to the subject for a single day followed by an interval period before the next dose. In some embodiments, the interval period is at least 3 days, 4 days, 5 days, 6 days, or 7 days.

In some embodiments, the pharmaceutical composition is administered after the interval period once daily for 14 days, 15 days, 16 days, 17 days, 18 days, 19 days, 20 days, 21 days, 22 days, 23 days, 24 days, 25 days, 26 days, 27 days, or 28 days.

In some embodiments, the pharmaceutical composition is formulated as a capsule or a tablet. In some embodiments, the pharmaceutical composition comprises an enteric coating or micro encapsulation. In some embodiments, the capsule is an enteric coated capsule.

In some embodiments, the subject is a mammal. In some embodiments, the subject is a human.

In certain embodiments, the pharmaceutical compositions and dosage forms, described herein can be administered in conjunction with any other conventional treatment. These treatments may be applied as necessary and/or as indicated and may occur before, concurrent with or after administration of the pharmaceutical compositions, dosage forms, and kits described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, and other compounds such as drugs being administered concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow, and replicate. The dose of the pharmaceutical compositions described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to delay onset of disease onset, or slow or stop its progression. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including hu0mans.

In accordance with the above, in therapeutic applications, the dosages of the active agents used in accordance with the invention vary depending on the active agent, the age, weight, and clinical condition of the recipient patient, and the experience and judgment of the clinician or practitioner administering the therapy, among other factors affecting the selected dosage.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a pharmaceutical composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response and/or the time period for a subject to clear the bacteria from normal tissue. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the bacteria from normal tissue; for example, the time period can be more than the time period for a subject to clear the bacteria from normal tissue, such as more than about a day, more than about two days, more than about three days, more than about five days, or more than about a week.

In some embodiments, the delivery of an additional therapeutic in combination with the pharmaceutical composition described herein reduces the adverse effects and/or improves the efficacy of the additional therapeutic.

The effective dose of an additional therapeutic described herein is the amount of the therapeutic agent that is effective to achieve the desired therapeutic response for a particular patient, composition, and mode of administration, with the least toxicity to the patient. The effective dosage level can be identified using the methods described herein and will depend upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of an additional therapy will be the amount of the therapeutic agent which is the lowest dose effective to produce a therapeutic effect. Such an effective dose will generally depend upon the factors described above.

The toxicity of an additional therapy is the level of adverse effects experienced by the subject during and following treatment. Adverse events associated with additional therapy toxicity include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heartbeat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

In certain embodiments, the therapeutic effects of these orally delivered medicines (e.g., pharmaceutical compositions) come from their action on pattern recognition receptors on immune cells in the lining of the small intestine. These cells, in turn, modulate immune cells circulating throughout the body. The medicines are microbes, but do not target the microbiome. In some embodiments, the microbes do not colonize or persist in the gut and do not modify the colonic microbiome. In some embodiments, they are gut-restricted. In some embodiments, the therapeutic effects of these orally delivered medicines are determined by examining for a biomarker measuring reaction of host (person) to infection (i.e., cytokine response, T cells and T cell ratios); an effect on infection itself (like virus measurement in sputum or swabs); or a clinical endpoint (like mortality or chest x-ray, clearance of virus).

In certain embodiments, the methods provided herein result in change (e.g., an increase or a decrease) in serum and/or expression levels of one or more cytokines (or one or more cellular factors) after the subject is treated according to a method provided herein for a set time interval as compared to before treatment and/or at the onset of treatment. In certain embodiments, the one or more cytokines (or one or more cellular factors) include TNF-α, IL-1β, IL-2, IL-6, IL-7, IL-10, IP10, MCP1, sIL-2R, IL-8, IL-1Ra, IL-2Ra, IL-18, HGF, MCP-1, MCP-3, MIG, M-CSF, GM-CSF, G-CSF, MIG-1a, and/or macrophage inflammatory protein (MIP)-lalpha (MIP1a). In certain embodiments, the one or more cytokines include TNF-α, IL-1β, IL-6, and/or IL-8. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days. The levels of the one or more cytokines can be determined, e.g., by ex vivo LPS stimulation of whole blood samples obtained from a subject, e.g., as described herein.

In certain embodiments, the methods provided herein result in change (e.g., an increase or a decrease) in serum and/or expression levels C-reactive Protein (CRP) after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in change (e.g., an increase or a decrease) in serum T cell count (e.g., CD4⁺ T cell count and/or CD8⁺ T cell count) after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in change (e.g., an increase or a decrease) in the proportion of CD4⁺CD3⁺ T cells to CD8⁺ CD3⁺ T cells after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in an increased virological clearance rate (e.g., increased clearance of SARS-CoV-2 in a subject with COVID-19). In some embodiments, the virological clearance rate is determined based on throat swabs, sputum, and/or lower respiratory tract secretions taken from a treated subject after treatment compared to before treatment after the subject is treated according to a method provided herein for a set time interval as compared to before treatment and/or at the onset of treatment. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days.

In certain embodiments, the methods provided herein result in reduction in level of viral nucleic acid and/or protein (e.g., SARS-CoV-2 nucleic acid and/or protein) present in a subject after treatment compared to before treatment after the subject is treated according to a method provided herein for a set time interval as compared to before treatment and/or at the onset of treatment. In some embodiments, the viral nucleic acid level is determined using RT-PCR. In some embodiments, the viral protein level is determined using an ELISA assay. In some embodiments, the time interval is up to 28 days. In certain embodiments, the time interval is about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days about 25 days, about 26 days, about 27 days, and/or about 28 days

In certain embodiments, the methods provided herein result in reduction in the time a treated subject spends in an intensive care unit (ICU) compared to untreated subjects. In certain embodiments, the time treated subjects spend in an ICU is reduced by at least 5%, 10%, 15%, 20%, 25%, 30%, 35%, 40%, 45%, 50%, 55%, 60%, 65%, 70%, or 75% compared to untreated subjects.

In certain embodiments, the methods provided herein result in reduction in ventilator requirements of treated subjects compared to untreated subjects. In certain embodiments, the time treated subjects spend on a ventilator is reduced by at least 5%, 10%, 15%, 20%, 25%, 30%, 35%, 40%, 45%, 50%, 55%, 60%, 65%, 70%, or 75% compared to untreated subjects.

In certain embodiments, the methods provided herein result in reduction in mortality of treated subjects compared to untreated subjects. In certain embodiments, the mortality of treated subjects is reduced by at least 5%, 10%, 15%, 20%, 25%, 30%, 35%, 40%, 45%, 50%, 55%, 60%, 65%, 70%, or 75% compared to untreated subjects.

In certain embodiments the methods provided herein result in reduced requirements for oxygen therapy, measured by the ratio of oxygen saturation (SpO2)/fraction of inspired oxygen (FiO2). In certain embodiments, the methods provided herein result in decreased symptom duration, reduced progression along the WHO scale of disease severity, and/or reduced mortality.

### Subjects

In certain aspects, the methods provided herein reduce IL-8, IL-6, IL-1β, and/or TNFα expression levels in a subject in need thereof (e.g., as compared to a standard). In some embodiments, the subject in need thereof suffers from an IL-8, IL-6, IL-1β, and/or TNFα mediated disease or condition. In some embodiments, the subject in need thereof has been infected with a virus (e.g., a respiratory virus). In certain embodiments, the virus is a coronavirus, an influenza virus, and/or a respiratory syncytial virus. In certain embodiments, the virus is a coronavirus such as MERS, SARS (such as SARS-CoV-2). In certain embodiments, the virus is a SARS virus. In certain embodiments, the virus is SARS-CoV-2. In some embodiments, the subject has COVID-19.

In certain aspects, provided herein is a method of treating cytokine storm (cytokine release syndrome) in a subject in need thereof. In some embodiments, the cytokine storm is due to elevation in IL-8, IL-6, IL-1β, and/or TNFα expression levels. In some embodiments, the subject in need thereof has been infected with a virus (e.g., a respiratory virus). In certain embodiments, the virus is a coronavirus, an influenza virus, and/or a respiratory syncytial virus. In certain embodiments, the virus is a coronavirus such as MERS, SARS (such as SARS-CoV-2). In certain embodiments, the virus is a SARS virus. In certain embodiments, the virus is SARS-CoV-2. In some embodiments, the subject has COVID-19.

In some embodiments, the subject in need thereof is present in, is traveling to, and/or has been in a region where viral infection (e.g., coronavirus infection, influenza virus infection, and/or a respiratory syncytial virus infection) is endemic. In certain embodiments, the subject in need thereof is present in, is traveling to, and/or has been in a region where SARS-CoV-2 infection is endemic.

In some embodiments, the subject has been exposed to a source infected with a coronavirus, an influenza virus, and/or a respiratory syncytial virus. In certain embodiments, the subject has been exposed to a source infected with a coronavirus such as MERS, SARS (such as SARS-CoV-2). In certain embodiments, the subject has been exposed to a source infected with SAR-CoV-2.

In certain embodiments, the subject has and/or is at an increased risk for a cardiovascular disease.

In some embodiments, the subject has and/or is at an increased risk for diabetes (e.g., type 2 diabetes).

In certain aspects, provided herein is a method of treating a viral infection in a subject in need thereof, comprising administering to the subject a Prevotella histicola strain comprising at least 99% genomic, 16S and/or CRISPR sequence identity to the nucleotide sequence of the Prevotella histicola Strain B (NRRL accession number B 50329), wherein a Type I interferon response is not reduced, e.g., as determined by IFNα or IFNβ levels.

In certain aspects, provided herein is a method of treating a viral infection in a subject in need thereof, comprising administering to the subject a Prevotella histicola strain comprising at least 99% genomic, 16S and/or CRISPR sequence identity to the nucleotide sequence of the Prevotella histicola Strain B (NRRL accession number B 50329), wherein IFNα and/or IFNβ levels are not reduced.

In certain aspects, provided herein is a method of reducing inflammatory cytokine expression (e.g., reducing IL-8, IL-6, IL-1β, and/or TNFα expression levels) in a subject in need thereof, wherein a Type I interferon response is not reduced, e.g., as determined by IFNα or IFNβ levels.

In certain aspects, provided herein is a method of reducing inflammatory cytokine expression (e.g., reducing IL-8, IL-6, IL-1β, and/or TNFα expression levels) in a subject in need thereof, wherein IFNα and/or IFNβ levels are not reduced.

In some embodiments, the subject in need thereof is a child (e.g., a child of no more than 10, 9, 8, 7, 6, 5, 4, 3, 2, or 1 years old). In certain embodiments, the subject is an infant of no more than 12, 11, 10, 9, 8, 7, 6, 5, 4, 3, 2, or 1 months old.

In certain embodiments, the subject is an older adult. In certain embodiments, the subject is at least 50, 55, 60, 65, 70, 75, 80, 80, or 90 years old.

In some embodiments, the subject is a pregnant woman. In some embodiments, the subject is a woman of child-bearing age.

In certain embodiments, the subject is immunocompromised (e.g., a subject who has undergone radiation therapy, immunotherapy, has received a transplant, is taking anti-rejection medication, is taking immunosuppressant medication, is infected with HIV, etc.).

In some embodiments, the subject treated according to the methods provide herein has an IL-8-mediated disease or condition. In certain embodiments, the IL-8 mediated disease or condition comprises Severe Acute Respiratory Syndrome (SARS), influenza, respiratory syncytial viral infection, atherosclerosis, melanoma, ovarian carcinoma, lung cancer, prostate cancer, gastric carcinoma, breast cancer, head-and-neck cancer, colon cancer, colitis-associated cancer, kidney cancer, pancreatic cancer, Crohn's disease (CD), Ulcerative Colitis (UC), Ischemia-Reperfusion injury (IRI), acute lung injury, asthma, chronic obstructive pulmonary disease (COPD), cystic fibrosis (CF), pulmonary fibrosis, multiple sclerosis, psoriasis, atopic dermatitis, rheumatoid arthritis, crescentic glomerulonephritis, IgA nephropathy, membranoproliferative glomerulonephritis, lupus nephritis, or membranous nephropathy, alcoholic hepatitis, or HIV-associated neurocognitive disorder. In certain embodiments, the IL-8 mediated disease or condition comprises a coronavirus, an influenza virus, and/or a respiratory syncytial virus. In certain embodiments, the IL-8 mediated disease or condition comprises a coronavirus such as MERS, SARS (such as SARS-CoV-2). In certain embodiments, the virus is a SARS virus. In certain embodiments, the virus is SARS-CoV-2. In some embodiments, the IL-8 mediated disease is COVID-19.

In some embodiments, the subject treated according to the methods provide herein has an IL-6 mediated disease or condition. In certain embodiments, the IL-6 mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-6 mediated disease or condition comprises a coronavirus, an influenza virus, and/or a respiratory syncytial virus. In certain embodiments, the virus is a SARS virus. In certain embodiments, the IL-6 mediated disease or condition comprises a coronavirus such as MERS, SARS (such as SARS-CoV-2). In certain embodiments, the virus is SARS-CoV-2. In some embodiments, the IL-6 mediated disease mediated disease is COVID-19.

In some embodiments, the subject treated according to the methods provide herein has an IL-1β mediated disease or condition. In certain embodiments, the IL-1β mediated disease or condition comprises Severe Acute Respiratory Syndrome (SARS), influenza, respiratory syncytial viral infection, Agammaglobulinemia, Amyloidosis, Ankylosing spondylitis, Anti-GBM/Anti-TBM nephritis, Antiphospholipid syndrome, Autoimmune hepatitis, Autoimmune inner ear disease, Atopic dermatitis, Asthma, Castleman disease, Celiac disease, Chagas disease, Chronic recurrent multifocal osteomyelitis, Cogan's syndrome, Cold agglutinin disease, CREST syndrome, Crohn's disease, Dermatomyositis, Devic's disease (neuromyelitis optica), Discoid lupus, Endometriosis, Eosinophilic esophagitis, Eosinophilic fasciitis, Evan's syndrome, Fibromyalgia, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Hypogammaglobulinemia, Hypoproliferative anemia, IgA Nephropathy, Inclusion body myositis, Interstitial cystitis, Inflammatory Bowel Disease, Juvenile arthritis, Juvenile/Type 1 Diabetes, Juvenile myositis, Kawasaki syndrome (Kawasaki Disease (and/or, e.g., Kawasaki disease shock syndrome (KDSS))), Lichen planus, Lichen sclerosis, Lupus (SLE), Meniere's disease, Multiple sclerosis, Myasthenia gravis, Microscopic polyangiitis, Optic neuritis, Pemphigus, Polyarteritis nodosa, Polymyalgia rheumatica, Polymyositis, Primary biliary cirrhosis, Primary sclerosing cholangitis, Psoriasis, Psoriatic arthritis, Rheumatic fever, Rheumatoid arthritis, Sarcoidosis, Sjogren's syndrome, Temporal arteritis/Giant cell arteritis, Transverse myelitis, Ulcerative colitis, Uveitis, Vasculitis, Vitiligo, Viral myocarditis, or Wegener's granulomatosis (Granulomatosis with Polyangiitis (GPA)). In certain embodiments, the IL-1β mediated disease or condition comprises a coronavirus, an influenza virus, and/or a respiratory syncytial virus. In certain embodiments, the IL-1β mediated disease or condition comprises a coronavirus such as MERS, SARS (such as SARS-CoV-2). In certain embodiments, the virus is a SARS virus. In certain embodiments, the virus is SARS-CoV-2. In some embodiments, the IL-1β mediated disease is COVID-19.

In some embodiments, the subject treated according to the methods provide herein has a TNFα mediated disease or condition. In some embodiments, the TNFα mediated disease or condition is Severe Acute Respiratory Syndrome (SARS), influenza, respiratory syncytial viral infection, rheumatoid arthritis, juvenile chronic arthritis, Crohn's disease (CD), Ulcerative Colitis (UC), ankylosing spondylitis, psoriasis, multiple sclerosis, atherosclerosis, myocardial infarction, heart failure, myocarditis, cardiac allograft rejection, asthma, ischemic renal injury, renal transplant rejection, glomerulonephritis, or inflammatory eye disease. In certain embodiments, the TNFα mediated disease or condition comprises a coronavirus, an influenza virus, and/or a respiratory syncytial virus. In certain embodiments, the TNFα mediated disease or condition comprises a coronavirus such as MERS, SARS (such as SARS-CoV-2). In certain embodiments, the virus is a SARS virus. In certain embodiments, the virus is SARS-CoV-2. In certain embodiments, the virus is SARS-CoV-2. In some embodiments, the TNFα mediated disease is COVID-19.

In some embodiments, the subject treated according to the methods provided herein has autoantibodies, e.g., autoantibodies against type I interferons (e.g., a higher amount of autoantibodies, e.g., than a standard). In some embodiments, the type I interferons are autoantibodies against type I IFN-α2 and/or IFN-ω. In some embodiments, the subject has low or undetectable serum IFN-α levels during acute COVID-19. See Bastard et al., Science 370:423 (2020).

In some embodiments, the subject treated according to the methods provided herein has impaired type I interferon (e.g., IFNα or IFNβ) production and/or activity (e.g., as compared to a standard). In some embodiments, the subject treated according to the methods provided herein has highly impaired type I interferon (e.g., IFNα or IFNβ) production and/or activity (e.g., as compared to a standard). In some embodiments, the subject has no IFNβ and low IFNα production and/or activity (e.g., as compared a standard). See Hadjadj et al., Science 369:718-724 (2020).

In some embodiments, the subject treated according to the methods provided herein has a polymorphism in STING (stimulator of interferon (IFN) genes, encoded by TMEM173) that leads to delayed activation and/or over-activation of the STING pathway (e.g., as compared to a standard). See Berthelot and Liote, EBioMedicine 56 (2020).

In some embodiments, the subject treated according to the methods provided herein has diminished and/or delayed IFN, production (e.g., as compared to a standard). In some embodiments, the subject treated according to the methods provided herein has diminished and/or delayed type I interferon production (e.g., as compared to a cohort control or reference value, e.g., to a standard). See Galani et al., Nature Immunology 22: 32-40 (2021).

In some embodiments, the subject treated according to the methods provided herein has SARS-CoV-2 M protein-mediated impairment (e.g., decreases) in type I and type III interferon production (e.g., as compared to production levels in the absence of COVID-19 infection, e.g., in a standard). In some embodiments, the impairment is due to SARS-CoV-2 M protein targeting of RIG-I/MDA-5 signaling. See Zheng et al., Signal Transduction and Targeted Therapy 5:299 (2020).

In some embodiments, the subject treated according to the methods provided herein has post-acute COVID-19. In some embodiments, the post-acute COVID-19 comprises ongoing symptomatic COVID-19 for people who still have symptoms between 4 and 12 weeks after the start of acute symptoms. In some embodiments, the post-acute COVID-19 comprises post-COVID-19 syndrome wherein subjects have symptoms for more than 12 weeks after the start of acute symptoms. See Venkatesan, The Lancet 9:129 (2021).

In some embodiments, the post-acute COVID-19 comprises gut dysbiosis. See Yeoh et al., Gut 0:1-9 (2021).

As used herein, the standard that is compared to can be a cohort control or reference value or a baseline value (e.g., as compared to a later time point).

### Cytokine Release Syndrome (CRS)

CRS occurs when large numbers of white blood cells, including B cells, T cells, natural killer cells, macrophages, dendritic cells, and monocytes are activated and release inflammatory cytokines, which activate more white blood cells in a positive feedback loop of pathogenic inflammation. See also, Moore et al., Science, 1 May 2020: Vol. 368:6490, pp. 473-474.

CRS or cytokine reactions can occur in a number of infectious diseases including, those associated with infection by COVID-19 (SARS-CoV-2), other coronaviruses, (e.g., SARS-CoV, MERS-CoV), Ebola virus, influenza, cytomegalovirus, variola and group A streptococcus, and sepsis due to infection.

CRS or cytokine reactions can occur in a number of other diseases including multiple sclerosis, pancreatitis, graft-versus-host disease (GVHD), autoimmune disease, acute respiratory distress syndrome (ARDS), multiple organ dysfunction syndromes (including, systemic inflammatory response (SIRS) and secondary hemophagocytic lymphohistiocytosis (sHLH)). CRS has been observed with chimeric antigen receptor (CAR-T) T cell therapy.

See also, Shimabukuro-Vornhagen et al., Journal for ImmunoTherapy of Cancer (2018) 6:56.

In some embodiments, CRS, and/or a condition (such as a viral infection) associated therewith, can be treated with a pharmaceutical composition and/or a solid dosage form and/or a method provided herein.

Acute lung injury (ALI) can be a common consequence of a cytokine storm in the lung alveolar environment. In some embodiments, ALI can be treated with a pharmaceutical composition and/or a method provided herein.

### Additional Cellular Factors

As described herein, the pharmaceutical compositions and methods provided herein can be used to reduce inflammatory cytokine expression (e.g., IL-8, IL-6, IL-1β, and/or TNFα expression) in a subject. For example, the pharmaceutical compositions and/or a solid dosage forms and/or methods provided herein can be used to treat diseases and conditions associated therewith.

The pharmaceutical compositions and/or a solid dosage forms and/or methods provided herein can be used to reduce the level of an interleukin, a chemokine, a colony stimulating factor, and/or a tumor necrosis factor (TNF). For example, in addition to IL-8, IL-6, IL-1β, and/or TNFα, the pharmaceutical compositions and methods provided herein can be used to reduce expression of IL-1Ra, IL-2Ra, IL-7, IL-18, HGF, MCP-1, MCP-3, MIG, M-CSF, GM-CSF, G-CSF, MIG-1a, IP-10, MCP-1, and/or macrophage inflammatory protein (MIP)-1 alpha.

The pharmaceutical compositions and methods provided herein can be used to change the level of TNF-α, IL-1β, IL-2, IL-6, IL-7, IL-10, GCSF, IP10, MCP1, MIP1α, sIL-2R, IL-6, and/or IL-8.

### EXAMPLES

### Example 1

### Prevotella histicola Strain B in Healthy Participants and Participants with Mild to Moderate Psoriasis or Mild to Moderate Atopic Dermatitis

Prevotella histicola Strain B (NRRL accession number B 50329) has recently completed a series of cohorts in a phase lb study in human volunteers and patients with psoriasis.

The primary endpoints were safety and tolerability. Prevotella histicola Strain B (NRRL accession number B 50329) has a placebo-like profile, consistent with the lack of systemic absorption. There was no persistence beyond the 28 day daily dosing period and no modification of the colonic microbiome by 16S RNA sequencing of patient stool samples.

Two cohorts of patients with mild-to-moderate psoriasis were treated with a low dose (1.6×10¹¹ cells per day) and high dose (8×10¹¹ cells per day) of Prevotella histicola Strain B (NRRL accession number B 50329) daily for 28 days. The lower dose was estimated by allometric scaling of the just-maximally effective dose in mouse inflammation models. The high dose was 5X× higher.

Whole blood samples were obtained from each subject. A whole blood stimulation assay was performed using sub-optimal LPS stimulus. Fresh sodium heparin anticoagulated bloods were collected from 12 subjects and couriered at ambient temperature. On receipt of samples, the blood was used to set up the following conditions: (a) Unstimulated and (b) LPS stimulated (10 ng/ml) at 24 h.

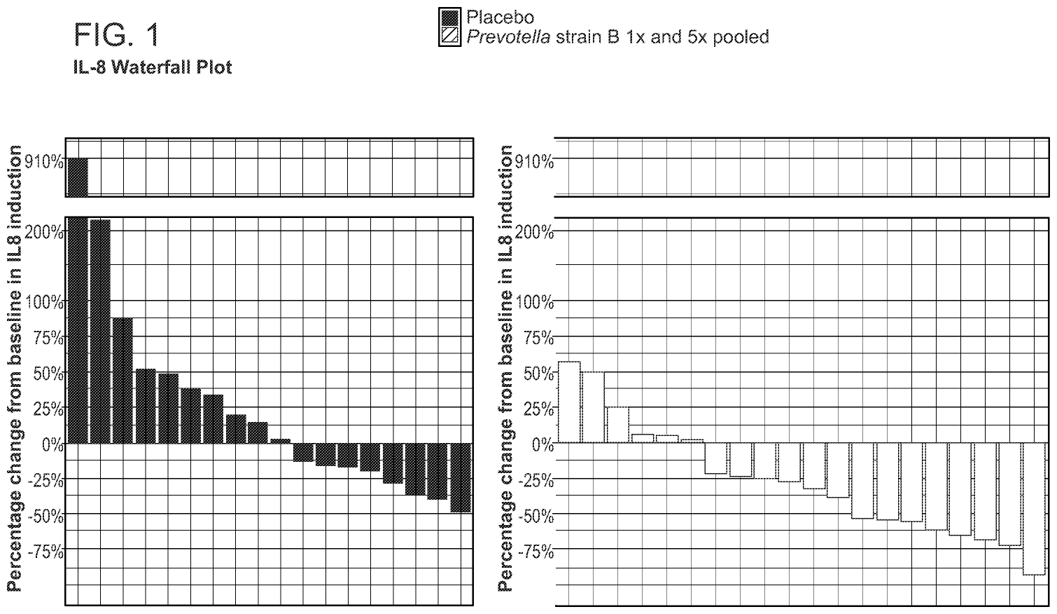

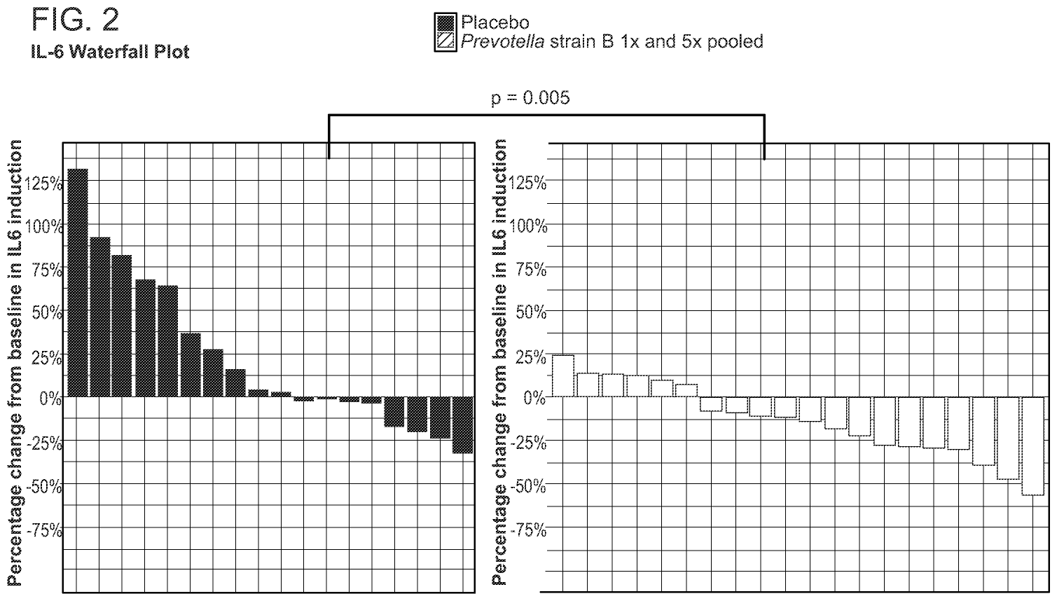

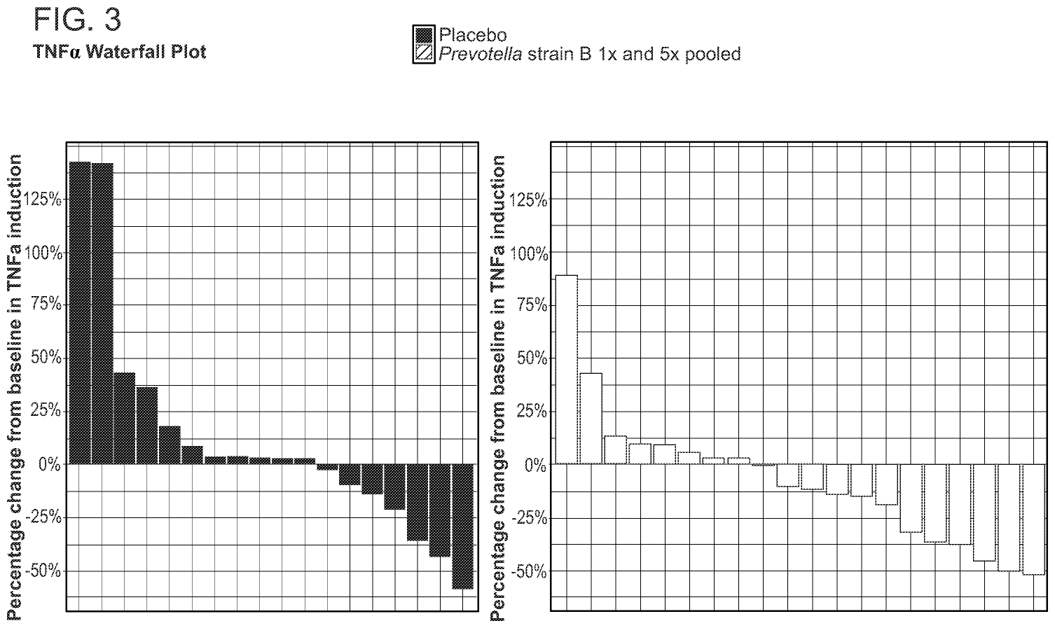

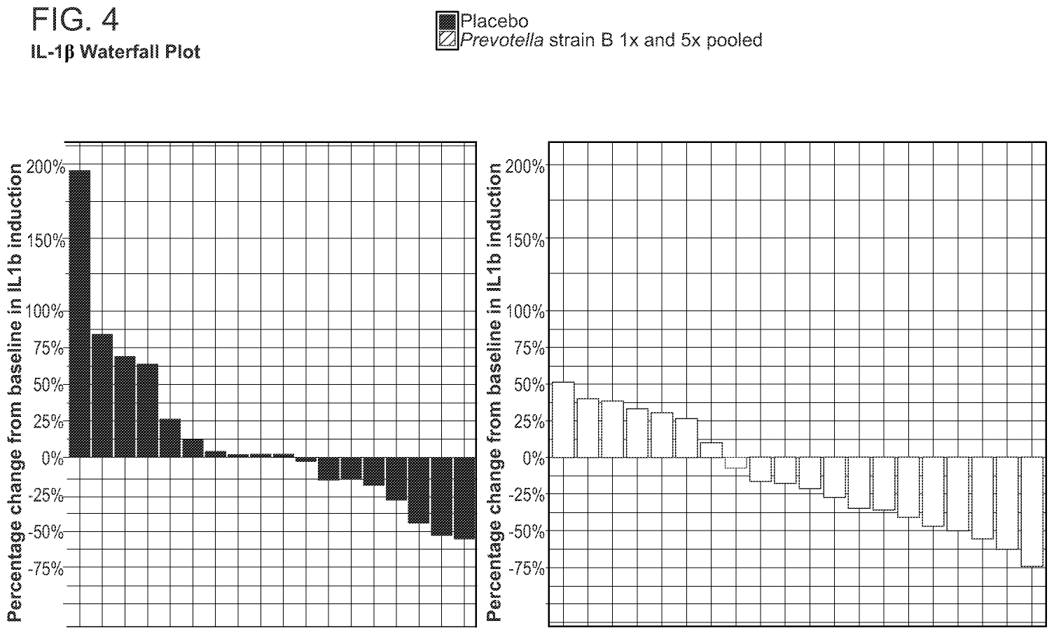

After incubation, plasma was isolated by centrifugation and stored at −80° C. All stored samples were evaluated for IL-6, IL-1β, IL-8, TNFα, IL-10, and IFNγ using Luminex. A total of four Luminex assays were performed. All samples from a single subject were run together on the same Luminex assay plate to avoid inter-assay variation when comparing baseline and last-dose samples. Waterfall plots illustrating the percent change in IL-6, IL-1β, IL-8, TNFα cytokine expression by subjects after 28 days of treatment with Prevotella histicola Strain B (right) or placebo (left) are provided in FIGS. 1-4: FIG. 1 (IL-8); FIG. 2 (IL-6); FIG. 3 (TNFα); FIG. 4 (IL-1β). Similar results were not seen for IL-1β or IFNγ.

### Example 2

### An Adaptive Phase 2 Double-Blind Placebo-Controlled Study Investigating Prevotella histicola Strain B in the Treatment of Pulmonary Complications of COVID-19 Rationale

As set forth herein, Prevotella histicola Strain B is an oral, potent and well-tolerated inhibitor of multiple systemic cytokines, including IL-6, IL-8 and TNFa (TNFα). Its unique profile and mechanism of action are not reflected in the current range of COVID-19 trials. IL-6, IL-8 and TNFa are key cytokines in adverse host response to infection. Clinical safety and tolerability are placebo-like. Administration of Prevotella histicola Strain B does not result in gut colonization or gut microbiome alteration.

An adaptive phase 2 double-blind placebo-controlled clinical study to assess safety and efficacy of Prevotella histicola Strain B in healthy participants and participants with either evidence of pulmonary involvement on hospitalization due to COVID-19 is performed. Daily doses include a 0.5× dose (8×10¹⁰ cells), a lx dose (1.6×10¹¹ cells) or a 5× dose (8×10¹¹ cells), each of which are compared to patients treated with placebo. Subjects are treated daily for up to 21 days or until resolution of pulmonary symptoms or progression to mechanical ventilation. The exploratory endpoints are designed to establish whether there are any effects on the systemic immune system and potential clinical benefit.

Participants who are successfully screened are randomized to either the active (Prevotella histicola Strain B) or placebo group on Day 1 and dosing is initiated. All safety data is reviewed in an ongoing and cumulative manner by the Principal Investigator (or delegate), Medical Monitor and the safety review committee (SRC).

### Design:

Up to 500 subjects with evidence of pulmonary involvement on hospitalization due to COVID-19 are randomized for treatment with Prevotella histicola Strain B or placebo plus best standard of care. The subjects may be older adults and/or patients with cardiovascular disease, diabetes, and or certain other pre-existing morbidities. Treatment is for up to 21 days or until resolution of pulmonary symptoms or progression to mechanical ventilation. An interim analysis for futility or continuation is carried out after 20 patients have been treated. The final numbers in the study is estimated from the interim analysis. Treatment is once a day Prevotella histicola Strain B at a dose of 8×10¹° cells (276 mg), 1.6×10¹¹ cells (550 mg), or 8×10¹¹ cells (2.76 g) administered as encapsulated freeze-dried powder or encapsulated enteric-coated mini-tablets, each containing 8×10¹° cells (276 mg) Prevotella histicola Strain B.

The effect of Prevotella histicola Strain B in subjects with COVID-19 is determined. The level of SARS-CoV-2 infection in the COVID-19 patients may be determined by RT-PCR. Categories of readouts include 1) biomarkers measuring reaction of host (person) to infection, i.e., cytokine response, T cells and T cell ratios; 2) effect on infection itself, like virus measurement in sputum or swabs; and 3) a clinical endpoint like mortality or chest x-ray, clearance of virus.

Blood samples, throat swabs, sputum and/or lower respiratory tract secretions are collected at various time points following initial treatment with Prevotella histicola Strain B to test markers of infection, immunology, and inflammation. The samples may be collected from baseline (0 week) to up to 12 weeks after treatment.

The change in serum expression levels of TNF-α, IL-1β, IL-2, IL-6, IL-7, IL-10, GSCF, IP10, MCP1, MIP1α, sIL-2R, IL-6, IL-8, or other cytokines after treatment compared to before treatment. Cytokine levels may be assessed using commercial ELISA methods. The change in expression levels of C-reactive Protein (CRP) or procalcitonin is also tested after treatment compared to before treatment.

The change in cell count of lymphocytes, CD4+ T cells, or CD8+ T cells is also tested after treatment compared to before treatment. The CD4+ T cells and CD8+ T cells may be counted via flow cytometry for peripheral whole blood. Lymphocyte count may be assessed by routinely used determination of blood count. The change in proportion of CD4⁺ CD3⁺ T cells to CD8⁺ CD3⁺ T cells is also tested after treatment compared to before treatment.

Further, the virological clearance rate of throat swabs, sputum, or lower respiratory tract secretions is tested after treatment compared to before treatment. For example, the change in levels of SARS-CoV-2 nucleic acid is determined after treatment compared to before treatment. The SARS-CoV-2 nuclei acid may be quantified using RT-PCR.

Other clinical readouts of infection severity are also determined. The time in the ICU, duration of hospitalization, ventilator requirements, and mortality rate are observed and recorded. For example, 1-month mortality is defined as the ratio of patients who will alive after 1 month from study start out of those registered at baseline. In addition, PaO2 (partial pressure of oxygen) FiO2 (fraction of inspired oxygen, FiO2) ratio (or P/F ratio) may be calculated from arterial blood gas analyses. Change of the SOFA (Sequential Organ Failure Assessment) is also assessed after treatment to evaluate 6 variables, each representing an organ system (one for the respiratory, cardiovascular, hepatic, coagulation, renal and neurological systems). Radiological response like thoracic CT scan or chest X-ray may also be performed.

### Example 3

### Effect of Prevotella histicola Strain B on Cytokine Expression in Preclinical Models

In preclinical models, Prevotella histicola Strain B has been observed to have effects on Th1, Th2, and Th17 pathways, including TNF, IL-5, IL-6, IL-12p40, IL-13, and IL-17. Several of these cytokines have been implicated in the cytokine storm associated with severe complications of COVID-19. In these models, no activity was observed on type 1 interferons, which are important for anti-viral responses.

### Example 4

### A Phase 2 Double-Blind Placebo-Controlled Study Investigating the Safety and Efficacy of Prevotella histicola Strain B in the Treatment of Patients Hospitalized with SARS-CoV-2 Infection

### Background

The COVID-19 pandemic, as declared on 11th Mar. 2020 by World Health Organization (WHO), is caused by a novel coronavirus (SARS-Cov-2). It is estimated to result in ˜50,000-160,000 deaths in the USA, if optimal healthcare can be delivered, and up to in excess of 2.2 million deaths if healthcare resources such as ventilated beds are exhausted (Cookson 2020). It is the pulmonary complications of the viral infection that results in the majority of hospitalizations, admissions to ICU and ultimately death (Guan 2020; Huang 2020; Liu 2020; Wang 2020). The COVID-Related Complications (CRC) include acute respiratory distress syndrome (ARDS), arrhythmia, shock, acute kidney injury, acute cardiac injury, liver dysfunction and secondary infection (Huang 2020; Maharaj 2020). There are no vaccines, prophylactic or therapeutic agents of proven efficacy. Significant symptoms that do not result in hospitalization are also common and result in significant illness even short of hospitalization.

Study of coronavirus infections in tissue culture and animal models and of historical, SARS-coronavirus outbreaks, provide insights into the likely pathophysiology of infection with COVID-19 (Guan 2020; Gralinski 2015). The majority of tissue damage following infection with SARS-Cov1 appears to be due to a later, exaggerated, host immune response (Gralinski 2015). The host anti-viral response is driven by the induction of type I interferons which inhibit transcription and translation of the viral genome and reduce the threshold for activation of natural killer cells. Type I interferons also decrease expression of Serping1, a regulator of the complement system and coagulation proteases; this may lead to complement-mediated tissue damage and a prothrombotic tendency. In airway epithelial cells, type I IFNs upregulate expression of ACE2 in airway epithelial cells. Whereas ACE2 has been shown to be protective in models of acute lung injury, it is also the receptor for the spike protein of COVID-19 and is used by the virus for binding to its target cells.

While SARS-CoV-2 infection evades detection by the immune system in the first 24 h of infection, after 7-14 days following symptom onset an exaggerated response from the host immune system occurs in a subgroup of people. This leads to progressive lung damage leading to the need for hospitalization and oxygen therapy that can progress to severe pulmonary complications requiring ventilation and even death. It is important to note that the development of Diffuse Alveolar Damage (DAD) is often independent of high-titer viral replication (Peiris 2003). Other end organ damage can also occur secondary to the host immune response. This abnormal immune and inflammatory response in affected lungs includes production of high levels of IL-6, IL-8, TNFα, IL-1β, influx of neutrophils and cytotoxic T cells. A Th₂ (IL4, IL13) response from alternatively-activated macrophages, and an associated profibrotic phenotype (including increased TGF β and PDGFα production) can lead to lung fibrosis and chronic sequelae (Ruan 2020). Activation of the coagulation cascade is associated with development of fibrin clots in the alveoli. IL- 6 and IL-8 are increased in subjects hospitalized with coronaviral infections (Mehta 2020). A therapeutic agent with anti-inflammatory effects across IL-6, IL-8 and TNFα could prevent this host immune mediated organ damage. The host immune response is clearly important in the initial anti-viral response of the host. A prolonged and exaggerated immune response as measured by these cytokines/chemokines is however associated with pulmonary complications, hospitalization and ultimately death. A therapeutic agent that does not abrogate the initial host anti-viral immune response but modulates the delayed excess immune response via multiple pathways, restoring a state of immune homeostasis, could offer significant clinical benefit to subjects with COVID-19 infections.

Rationale: Prevotella histicola Strain B is a single strain of a small-intestine targeted, systemically immune-modulating, monoclonally-expanded commensal gut bacteria. Preclinical and clinical data have demonstrated that Prevotella histicola Strain B reduces levels of IL-6, TNFα and IL-8, while elevating epithelial expression of IL-1β and FoxP3. At the same time, Prevotella histicola Strain B is well tolerated with no overall difference from placebo in human trials to date. This profile could be highly relevant with respect to treating COVID-Related Complications (CRC).

An exaggerated host immune response leads to the life-threatening complications of COVID-19 infection. The cytokine IL-6 and chemokine IL-8 have been shown to be increased in subjects hospitalized with coronaviral infections, infections with influenza A, and in secondary HLH, and their exaggerated levels are pathogenic in the development of complications such as ARDS. The host immune response is clearly important in the initial anti-viral response of the host and IL-6 in particular has been shown to be important in the early phase of the infection. A prolonged and exaggerated immune response is however associated with pulmonary complications, hospitalization and ultimately death. A therapeutic agent that does not abrogate the host immune response entirely, but instead modulates multiple pathways and returns it back to a state of immune homeostasis, could offer significant clinical benefit to subjects with coronaviral infections.

The profile of Prevotella histicola Strain B as an oral agent with good tolerability modulating multiple key immune pathways without blocking them completely—that is, immune normalization rather than immune suppression—could offer significant clinical benefit to patients at risk of developing serious complications secondary to COVID-19.

TABLE 1
Clinical Trial Objectives and Endpoints

| Objectives | Endpoints |
|---|---|
| Primary | |
| To evaluate the effect of Prevotella | Change from baseline to the lowest S/F ratio |
| histicola Strain B on pulmonary function | measured in days 1-14 |
| as measured by the change in Oxygen | |
| Saturation (SpO2)/Fraction of Inspired | |
| Oxygen (FiO2) [S/F] ratio | |
| Secondary | |
| To evaluate the effect of Prevotella | Change in S/F ratio at days 4, 7, 10 and |
| histicola Strain B on the development | 14/discharge day. |
| and severity of complications of COVID- | Percentage change in S/F ratio at days 4, 7, |
| 19 infection | 10 and 14/discharge day. |
| | Percentage of participants at each level on |
| | the WHO OSCI score at days 4, 7, 14, 21 |
| | and 42. |
| | Percentage of participants with shifts from |
| | each level of the WHO OSCI score at |
| | baseline at days 4, 7, 14, 21 and 42. |
| | Percentage of participants remaining at their |
| | baseline score on the WHO OSCI (or lower) |
| | at days 4, 7, 14, 21 and 42. |
| | Percentage of participants reporting each |
| | level of the WHO OSCI score at their worst |
| | post-baseline day. |
| | The time in days spent at each participant's |
| | worst reported WHO OSCI score |
| | (excluding death). |
| | Intubation and mechanical-ventilation free |
| | survival, defined as the time in days from |
| | start of treatment to first occurrence of a |
| | WHO OSCI score of 6 or more. |
| | Overall survival, defined as the time in days |
| | from start of treatment to death by any |
| | cause |
| | Number of days requiring oxygen therapy. |
| | Number of days with pyrexia ≥38° C. |
| | Maximum daily temperature. |
| | Minimum SpO2 level. |
| | Maximum SpO2 level. |
| To evaluate the effect of Prevotella | Time to discharge, defined as the time in |
| histicola Strain B on length of | days from start of treatment to first |
| hospitalization and recovery in | occurrence of a WHO OSCI score of 2 or |
| participants with COVID-19 | less. |
| | Time to oxygen saturation (SpO2) ≥94% on |
| | room air without further requirement for |
| | oxygen therapy. |
| | Time to recovery, defined as the time in |
| | days from symptom onset to alleviation of |
| | all COVID-19 symptoms. |
| To evaluate the safety and tolerability | Incidence and severity of treatment |
| of Prevotella histicola Strain B in | emergent adverse events and serious |
| participants with COVID-19 | adverse events. |
| | Incidence of clinically significant abnormal |
| | changes in safety laboratory parameters. |
| Exploratory | |
| To evaluate the effect of Prevotella | Change from baseline in cytokine levels |
| histicola Strain B on the exaggerated | (including IL-6) at day 4 and day 7 (and/or |
| host cytokine response to COVID-19 | at additional timepoints if samples are |
| infection | available). |
| | Change from baseline in inflammatory |
| | response at day 4 and day 7 (and/or at |
| | additional timepoints if samples are |
| | available). |
| To identify clinical or biochemical | Statistical significance of each potential |
| predictors of response to Prevotella | predictor-treatment interaction in |
| histicola Strain B, and to confirm lack | exploratory models based on the final |
| of systemic absorption. | selected model for the primary efficacy |
| | analysis. |
| | To identify the presence of Prevotella |
| | histicola Strain B in stool and/or blood |
| | using PCR primers |

### Overall Design:

This is a randomized, placebo-controlled clinical study to assess the safety and efficacy of Prevotella histicola Strain B in patients hospitalized with COVID-19 infection. The study is designed to evaluate the efficacy of Prevotella histicola Strain B at reducing time to resolution of symptoms, preventing progression of COVID-19 symptoms and preventing COVID-Related Complications (CRC). The study will be fully blinded to the participants, investigator, and sponsor. This is a pilot study with a primary objective of investigating the potential of Prevotella histicola Strain B in the prevention of COVID-19 disease progression. The secondary objective is to evaluate multiple endpoints for clinical relevance and sensitivity, while informing the sample size for future studies. Where possible, data will be taken from assessments performed as part of the participant's routine clinical care in this pragmatic study.

Participants who are hospitalized with confirmed COVID-19 disease and are confirmed to be eligible for the study will be randomized to either the active (Prevotella histicola Strain B) or placebo group (1:1 randomization), in addition to standard of care. Dosing will be initiated on a twice daily regime for the first 3 days (6 doses) and then once daily for the remaining 11 days (14 days total treatment course). The trial hypothesis is that treatment with Prevotella histicola Strain B in hospitalized patients reduces oxygen requirements by normalizing the exaggerated host immune response to COVID-19. This will be measured by assessing the ratio of the Oxygen Saturation (SpO2)/Fraction of Inspired Oxygen (FiO2), which is a validated measure of severity of ARDS.

Dosing will be stopped if participants are admitted to ICU, efficacy and safety data will continue to be collected according to the schedule of activities, where practical. However, if the participant is eligible for another interventional trial at this point, they may be enrolled into it, and withdrawn from this study, after discussion with the chief investigator. Inclusion in concurrent interventional studies will not be permitted. Inclusion in observational studies in parallel to this study is allowed.

### Participant Trial Duration:

This study will consist of a 14-day treatment period followed by a 28-day post-treatment follow-up visit.

### Dose Justification:

Prevotella histicola Strain B will be administered as an enteric coated powder in capsule formulation.

The treatment regimen for this study will be 1.6×10¹¹ cells of Prevotella histicola Strain B (2 capsules) given twice a day for 3 days (6 doses) then once a day for 11 days (14-day total course).

The doses of Prevotella histicola Strain B tested in humans to date (1.6×10¹¹ cells to 8.0×10¹¹ cells) were based on allometric scaling from the preclinical in vivo experimental data. Both doses had clear effects on IL-6 and IL-8 based on lipopolysaccharide (LPS) stimulation of whole blood samples taken at baseline and after a course of daily administration of Prevotella histicola Strain B. The study included patients with mild to moderate psoriasis, and improvements in their skin condition were also demonstrated. There was no clear difference between the 2 dose levels tested on IL-6 and IL-8 and so the lower dose has been selected for this study.

An initial twice daily (bd) dosing regimen has been selected to maximize the speed of response. Prevotella histicola Strain B works via direct interaction with immune cells in the epithelium of the upper small intestine. A twice a day regimen doubles the duration of exposure of the microbes to the immune cells in the upper small intestine per 24 hours and will increase the speed of response.

### Study Population:

This protocol contains participants with a confirmed diagnosis of COVID-19 viral infection.

### Drug Product:

The Prevotella histicola Strain B drug product is available as enteric coated hydroxylpropyl methylcellulose (HPMC) hard capsules in white to off-white color. The formulation of Prevotella histicola Strain B consist of freeze-dried powder of P histicola and excipients. The excipients include mannitol, magnesium stearate and colloidal silicon dioxide. Each Prevotella histicola Strain B powder in capsule (PIC) contains 8.0×10¹⁰ cells of P histicola.

Treatment with Prevotella histicola Strain B or placebo will be twice daily for 6 doses and then once daily for 11 doses (14-day total course). There should be a minimum of two hours between the twice daily doses. For subjects who are discharged within the 14-day period, medication will be dispensed to take at home.

### Efficacy Assessments:

Oxygen Saturation: Oxygen saturation will be measured using a peripheral pulse oximeter and will also be analyzed as a ratio with the oxygen flow (SpO₂/FiO₂). The measurement will ideally be performed with the subject sitting and having been rested for at least 10 minutes.

If the subject is on 3 litres/min oxygen flow or less, and the investigator feels it is safe to do so, the investigator will remove the subject's supplemental oxygen for 10 minutes while they remain seated, and while continuously monitoring the oxygen saturation. After 10 minutes the oxygen saturation reading will be taken to calculate the S/F ratio on room air. If, during this process, the saturations drop by greater than 4%, the oxygen will be immediately replaced and the ratio measured on oxygen.

WHO Ordinal Scale: The WHO ordinal scale (Table 2) will be collected throughout the study. This is an accepted instrument which has been developed specifically for trials in patients with COVID-19.

TABLE 2
WHO ordinal scale for clinical improvement (OSCI) of COVID-19

| Patient State | Descriptor | Score |
|---|---|---|
| Uninfected | No clinical or virological | 0 |
| | evidence of infection | |
| Ambulatory | No limitation of activities | 1 |
| | Limitation of activities | 2 |
| Hospitalized | Hospitalized, no oxygen therapy | 3 |
| Mild disease | Oxygen by mask or nasal prongs | 4 |
| Hospitalized | Non-invasive ventilation or | 5 |
| Severe Disease | high-flow oxygen | |
| | Intubation and mechanical ventilation | 6 |
| | Ventilation + additional organ | 7 |
| | support − pressors, RRT, ECMO | |
| Dead | Death | 8 |

### Biomarkers:

Biomarker samples will be collected at baseline, day 4 and day 7. A small panel of biomarkers will be conducted on all subjects at these time points. Additional biomarkers may be measured based on the results of the trial.

### Biomarkers to Be Measured on All Subjects:

Specific biomarkers have been associated with progression and poor outcome following infection with COVID-19. These include differential white cell count, neutrophil to lymphocyte ratio, CRP, IL-6, IL-8, Ferritin, D-Dimer, and Troponin levels. These will be measured in all subjects at baseline, day 4 and day 7.

### Additional Plasma Biomarkers:

Additional plasma biomarkers may be analyzed subject to the clinical data in the trial. These biomarkers may help understand the response to Prevotella histicola Strain B and/or the progression of COVID-19 disease. These markers could include Eotaxin, Eotaxin-3, GM-CSF, IFN-γ, IL-1α, IL-1β, IL-2, IL-4, IL-5, IL-7, IL-8 (HA), IL-10, IL-12/IL-23p40, IL-12p70, IL-13, IL-15, IL-16, IL-17A, IP-10, MCP-1, MCP-4, MDC, MIP-1α, MIP-1β, TARC, TNF-α, TNF-β, VEGF-A. Additional plasma biomarkers may be analyzed if emerging data suggests they could be useful in understanding the drug response and/or disease progression.

### Transcription Analysis:

RNA will be collected from PBMCs and may be analyzed subject to the clinical data in the trial. The exact genes to be analyzed will be defined by an expert sub-group of the study but will include genes related to host immune response as well as those related to the disease pathology.

### Microbiome Research:

The microbiome composition of stool samples will be assessed as an optional research test at baseline and day 7. Prevotella histicola Strain B is not expected to alter the composition of the microbiome, but the microbiome will be evaluated for separate research purposes. Microbiome analysis may be performed through 16s ribosomal RNA sequencing and/or whole genome microbial sequencing depending on the question being asked.

### REFERENCES

Cookson C. UK's original coronavirus plan risked ‘hundreds of thousands’ dead. Financial Times. 16 Mar. 2020.
Fine J P and Gray R J. A Proportional Hazards Model for the Subdistribution of a Competing Risk. J Amer Stat Assn, vol. 94, no. 446, 1999, pp. 496-509.
Gralinski L E, and Baric R S. Molecular pathology of emerging coronavirus infections. J Pathol, 2015: 235: 185-195.
Guan W J, Ni Z Y, Hu Y et al. Clinical Characteristics of Coronavirus Disease 2019 in China. NEJM, 2020: 382(18):1708-1720.
Hagau N, Slavcovici A, Gonganau D N et al. Clinical aspects and cytokine response in severe H1N1 influenza A virus infection. Crit Care, 2010: 14(6) R203.
Huang C, Wang Y, Li X et al. Clinical features of patients infected with 2019 novel coronavirus in Wuhan, China. Lancet. 2020; 395(10223):497-506.
de Jong M D, Simmons C P, Thanh T T et al. Fatal outcome of human influenza A (H5N1) is associated with high viral load and hypercytokinemia. Nat Med, October 2006: 12(10) 1203-7.
Maharaj R. King's Critical Care—Evidence Summary Clinical Management of COVID-19. King's Critical Care. 9 Mar. 2020: 1-24.
Liu Y, Yang Y, Zhang C et al. Clinical and biochemical indexes from 2019-nCoV infected patients linked to viral loads and lung injury. Sci. China Life Sci. 2020: 63, 364-374.
Mehta P, McAuley D F, Brown M et al. Covid-19: consider cytokine storm syndromes and immunosuppression; The Lancet. 2020: 395, 1033-4.
Peiris J S, Chu C M, Cheng V C et al. Clinical progression and viral load in a community outbreak of coronavirus-associated SARS pneumonia: a prospective study. Lancet. 2003: 361: 1767-72.
Ruan Q, Yang K, Wang W et al. Clinical predictors of mortality due to COVID-19 based on an analysis of data of 150 patients from Wuhan, China. Intensive Care Med. 3 Mar. 2020.
Villar J, Perez-Mendez L, Blanco J, et al. Spanish Initiative for Epidemiology, Stratification, and Therapies for ARDS (SIESTA) Network. A universal definition of ARDS: the PaO2/FiO2 ratio under a standard ventilatory setting—a prospective, multicenter validation study. Intensive Care Med. 2013 April;39(4):583-92.

Wang D. Hu B, Hu C et al. Clinical Characteristics of 138 Hospitalized Patients With 2019 Novel Coronavirus—Infected Pneumonia in Wuhan, China. JAMA. 2020: 323, 1061-1069.
Wong CbK, Lam C W K, Wu A K L et al. Plasma inflammatory cytokines and chemokines in severe acute respiratory syndrome. Clin Exp Immunol. 2004: 136, 95-103.

### Example 5

### Prevotella histicola Strain B Does Not Inhibit Type I Interferon Production in Preclinical Models

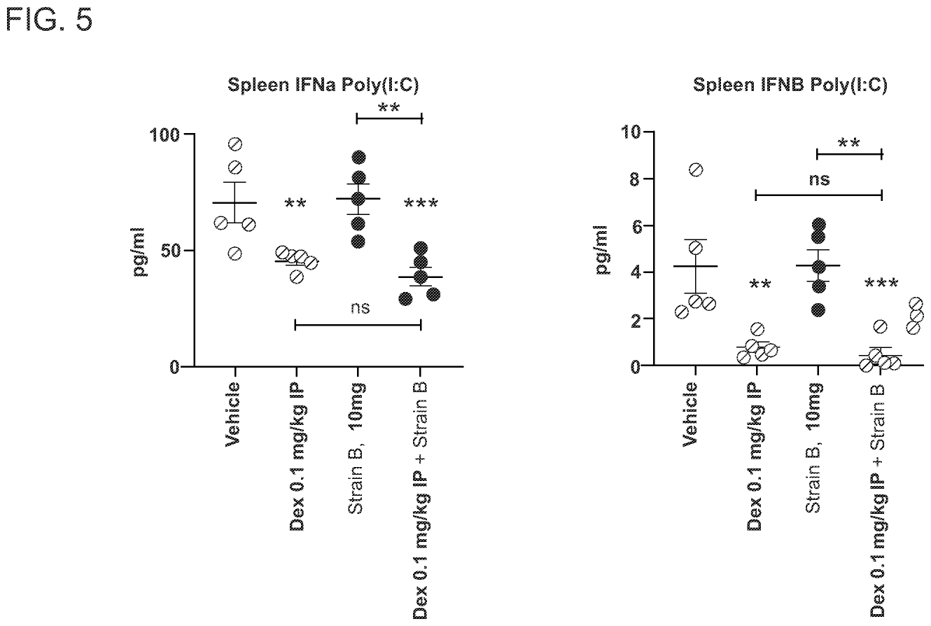

Evidence for the lack of immunosuppression comes from effects on type 1 interferons, as shown in FIG. 5. Spleen cells were removed from animals treated with Prevotella histicola Strain B or dexamethasone. The effect of the treatments on virally-induced production of interferons was mimicked by treating the cells with poly (I:C), an analog of double-stranded RNA. Prevotella histicola Strain B had no effect on IFNα or β, unlike dexamethasone which suppressed both, even at this sub-therapeutic dose. It was notable that while dexamethasone significantly inhibited the production of interferon-alpha and interferon-beta in the spleen cell stimulation assay (FIG. 5), Prevotella histicola Strain B mono-therapy had no impact on these Type 1 interferons. This demonstrates that Prevotella histicola Strain B can selective inhibit inflammation and pro-inflammatory cytokines, while preserving protective Type 1 interferon responses.

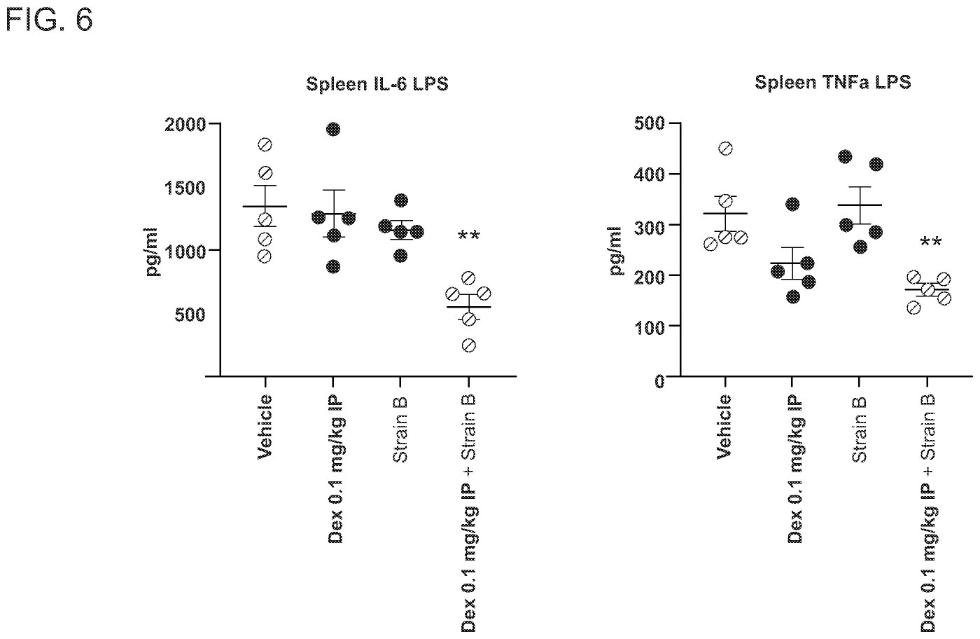

The combination significantly reduced the production of IL-6 and TNFα from spleen cells (FIG. 6).

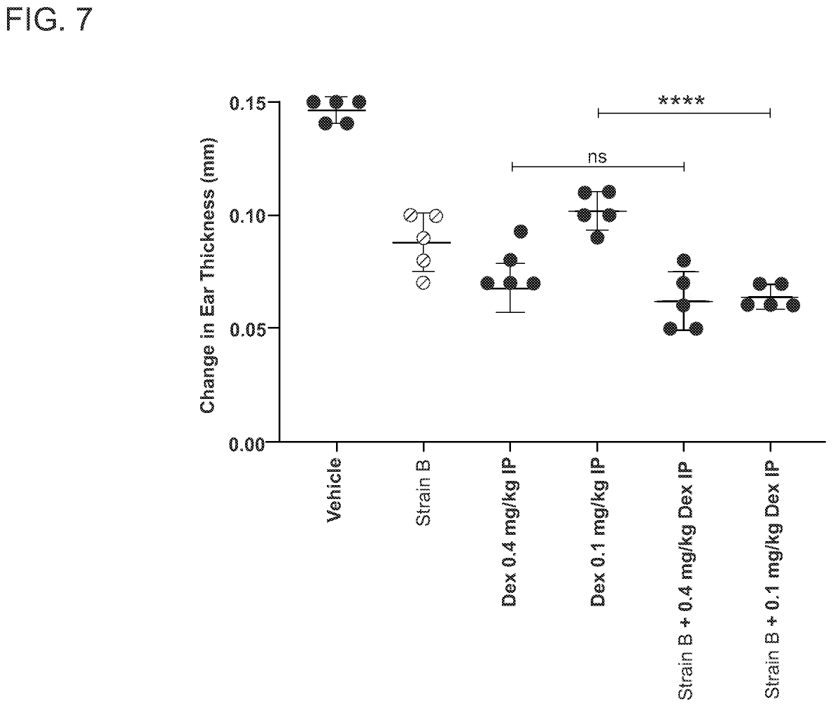

Three days of dosing with Prevotella histicola Strain B, one of two doses of dexamethasone, and the combination of Prevotella histicola Strain B with dexamethose all inhibited ear inflammation (FIG. 7). A dose-response relationship was seen with dexamethasone, and the combination of Prevotella histicola Strain B with the lower 0.1 mg/kg dose of dexamethasone was more efficacious than either Prevotella histicola Strain B or 0.1 mg/kg dexamethasone alone. This result suggests that Prevotella histicola Strain B may increase the efficacy of lower doses of corticosteroids, resulting in the reduction of the undesirable side effects associated chronic steroid use.

Methods: Mice were immunized by subcutaneous injection with KLH emulsified with Complete Freund's Adjuvant. On Day 6 after the sensitization, mice were dosed for 3 days with oral Prevotella histicola Strain B, dexamethasone (0.1 mg/kg or 0.4 mg/kg) given intraperitoneally, or a combination of Prevotella histicola Strain B and dexamethasone. One day 8, mice were challenged by intradermal ear injection with KLH. The DTH response was evaluated 24 hours post-challenge. For the ex vivo cytokine analysis, spleen cells from treated mice were incubated for 48 hours in vitro and stimulated with either LPS or polyinosinic-polycytidylic acid (poly I:C), a potent ligand for Toll-like receptor 3, which induces interferon-alpha (IFNα) and interferon-beta (IFNβ) from immune cells.

### Example 6

### Tablets

Examples of tablets that can be used include the following:

Tableting was performed and manufactured batches were first sub-coated with

Opadry QX blue before top-coating for enteric release with Kollicoat MAE100P.

TABLE 3
Prevotella histicola Tablet Composition

| | Active Dose |
|---|---|
| Material | (% w/w) |
| Prevotella histicola Strain B (NRRL accession | 25.0 |
| number B 50329) powder | |
| Mannitol 200 SD | 19.5 |
| L-HPC (LH-B1) | 32.0 |
| Crospovidone (Kollidon CL-F) | 15.0 |
| Croscarmellose Sodium (Ac-Di-Sol SD-711) | 6.0 |
| Colloidal Silica (Aerosil 200) | 1.0 |
| MG Stearate | 1.5 |
| Total | 100.0 |

The Prevotella histicola strain referred to above has been deposited as Prevotella histicola Strain B (NRRL accession number B 50329). The dose composition of Table 3 was provided in a 17.4 mm×7.1 mm tablet.

TABLE 4
Sub-coating Composition

| Materials | (% w/w) |
|---|---|
| Opadry QX Blue | 15.00 |
| WFI | 85.00 |
| Total | 100.00 |

TABLE 5
Top-coating Composition

| Materials | (% w/w) |
|---|---|
| Kollicoat MAE 100P | 15.00 |
| TEC | 2.25 |
| Talc | 3.00 |
| Water | 79.75 |
| Total | 100 |

The target weight per tablet is 650 mg (dose strength 162.5 mg).

As another example, the following recipe in Table 6 is prepared.

TABLE 6
Prevotella histicola Tablet Composition

| | Active Dose |
|---|---|
| Material | (% w/w) |
| Prevotella histicola Strain B (NRRL accession | 23.0 |
| number B 50329) powder | |
| Mannitol 200 SD | 21.5 |
| L-HPC (LH-B1) | 32.0 |
| Crospovidone (Kollidon CL-F) | 15.0 |
| Croscarmellose Sodium (Ac-Di-Sol SD-711) | 6.0 |
| Colloidal Silica (Aerosil 200) | 1.0 |
| MG Stearate | 1.5 |
| Total | 100.0 |

The tablet is prepared as a 17.4 mm×7.1 mm tablet. The tablet is enteric coated. The tablet contains 3.2×10¹¹ TCC of Prevotella histicola Strain B (NRRL accession number B 50329). The Prevotella histicola strain referred to above has been deposited as Prevotella histicola Strain B (NRRL accession number B 50329).

### Example 7

### Capsules

Examples of capsules that can be used include the following:

TABLE 7
Prevotella histicola Capsule Composition

| | | 1.6 × 10¹⁰ | 8.0 × 10¹⁰ | 1.6 × 10¹¹ |
|---|---|---|---|---|
| | | Cells | Cells | Cells |
| Name of ingredient(s) | Function | % w/w | % w/w | % w/w |
| Prevotella histicola | Active | 13.51 ^{b} | 90.22 ^{b} | 50 |
| (lyophilized) powder | ingredient | | | |
| Mannitol | Diluent | 84.99 ^{b} | 8.28 ^{b} | 48.5 |
| Magnesium Stearate | Lubricant | 1.0 | 1.0 | 1.0 |
| Colloidal Silicon | Glidant | 0.5 | 0.5 | 0.5 |
| Dioxide | | | | |
| Total Fill Weight | | 100 | 100 | 100 |
| Capsules^{a}, Size 0 | Capsule | 1 unit | 1 unit | 1 unit |
| | Shell | | | |

^{a}Composed of hydroxypropyl methylcellulose and titanium dioxide.
^{b} Adjusted based on the potency of drug substance to ensure targeted strength.

The Prevotella histicola strain referred to above has been deposited as Prevotella histicola Strain B (NRRL accession number B 50329). The capsule was banded with an HPMC-based banding solution. The banded capsule was enteric coated with a poly(methacrylic acid-co-ethyl acrylate) copolymer.

As another example, capsules according to the following recipe in Table 8 were prepared:

TABLE 8
Prevotella histicola Capsule Composition

| | | 3.35 × 10¹¹ |
|---|---|---|
| | | Cells |
| Name of ingredient(s) | Function | % w/w |
| Prevotella histicola | Active ingredient | 50 |
| (lyophilized) powder | | |
| Mannitol (Pearlitol SD200) | Diluent | 48.5 |
| Magnesium Stearate | Lubricant | 1.0 |
| (Ligamed MF-2-V) | | |
| Colloidal Silicon Dioxide | Glidant | 0.5 |
| (Aerosil 200P) | | |
| Total Fill Weight | | 100 |
| Capsules^{a}, Size 0 | Capsule Shell | 1 unit |

^{a}Swedish orange Vcap capsules

The Prevotella histicola strain referred to above has been deposited as Prevotella histicola Strain B (NRRL accession number B 50329). The capsule was banded with an HPMC-based banding solution. The banded capsule was enteric coated with Eudragit L30-D55, a poly(methacrylic acid-co-ethyl acrylate) copolymer.

### Incorporation by Reference

All publications and patent applications mentioned herein are hereby incorporated by reference in their entirety as if each individual publication or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of reducing IL-8, IL-6, IL-1β, and/or TNFα expression levels in a human subject in need thereof, comprising orally administering to the subject a Prevotella histicola strain comprising at least 99% genomic, 16S and/or CRISPR sequence identity to the nucleotide sequence of the Prevotella Strain B 50329 (NRRL accession number B 50329).

2. A method of treating a viral infection in a subject comprising orally administering to the subject a Prevotella histicola strain comprising at least 99% genomic, 16S and/or CRISPR sequence identity to the nucleotide sequence of the Prevotella Strain B 50329 (NRRL accession number B 50329).

3. A method of treating COVID-19 in a subject comprising orally administering to the subject a Prevotella histicola strain comprising at least 99% genomic, 16S and/or CRISPR sequence identity to the nucleotide sequence of the Prevotella Strain B 50329 (NRRL accession number B 50329).

4. The method of any one of claims 1 to 3, wherein the Prevotella histicola strain is the Prevotella Strain B (NRRL accession number B 50329).

5. The method of any one of claims 1 to 4, wherein the subject is administered at least 8×10¹⁰ total cells of the Prevotella histicola strain.

6. The method of any one of claims 1 to 4, wherein the subject is administered at least 1.6×10¹¹ total cells of the Prevotella histicola strain.

7. The method of any one of claims 1 to 4, wherein the subject is administered at least 8×10¹¹ total cells of the Prevotella histicola strain.

8. The method of any one of claims 1 to 4, wherein the subject is administered between 8×10¹⁰ and 8×10¹¹ total cells per day of the Prevotella histicola strain.

9. The method of any one of claims 1 to 4, wherein the subject is administered between 1.6×10¹¹ and 8×10¹¹ total cells per day of the Prevotella histicola strain.

10. The method of any one of claims 1 to 4, wherein the subject is administered about 8×10¹⁰ total cells per day of the Prevotella histicola strain.

11. The method of any one of claims 1 to 4, wherein the subject is administered about 1.6×10¹¹ total cells per day of the Prevotella histicola strain.

12. The method of any one of claims 1 to 4, wherein the subject is administered about 8×10¹¹ total cells per day of the Prevotella histicola strain.

13. The method of any one of claims 1 to 12, wherein the Prevotella histicola strain is administered in the form of one or more enteric-coated capsules.

14. The method of claim 13, wherein the capsule comprises a pH-sensitive enteric coating that such that the contents of the capsule are released into the human subject's duodenum and jejunum.

15. The method of any one of claims 1 to 12, wherein the Prevotella histicola strain is administered in the form of one or more enteric coated tablets.

16. The method of any one of claims 1 to 12, wherein the Prevotella histicola strain is administered in the form of one or more enteric-coated mini-tablets.

17. The method of claim 16, wherein the one or more enteric-coated mini-tablets are administered in one or more a non-enteric-coated capsules.

18. The method of any one of claims 1 to 17, wherein the subject is orally administered a plurality of doses of the Prevotella histicola strain.

19. The method of claim 18, wherein the human subject is administered daily doses of the Prevotella histicola strain for at least 7 days.

20. The method of claim 18, wherein the human subject is administered daily doses of the Prevotella histicola strain for at least 14 days.

21. The method of claim 18, wherein the human subject is administered daily doses of the Prevotella histicola strain for at least 28 days.

22. The method of claim 18, wherein the human subject is administered twice-daily doses of the Prevotella histicola strain.

23. The method of claim 22, wherein the human subject is administered twice-daily doses of the Prevotella histicola strain for at least 7 days.

24. The method of claim 22, wherein the human subject is administered twice-daily doses of the Prevotella histicola strain for at least 14 days.

25. The method of claim 22, wherein the human subject is administered twice-daily doses of the Prevotella histicola strain for at least 28 days.

26. The method of any one of claims 1 to 25, wherein the subject has been infected with a coronavirus, an influenza virus, and/or a respiratory syncytial virus.

27. The method of any one of claims 1 to 26, wherein the subject has been infected with SARS-CoV-2.

28. The method of claim 27, wherein the subject has COVID-19.

29. The method of any one of claims 1 to 28, wherein the subject is traveling to a region where SARS-CoV-2 infection is endemic.

30. The method of any one of claims 1 to 29, wherein the subject has been exposed to a source infected with a coronavirus, an influenza virus, and/or a respiratory syncytial virus.

31. The method of any one of claims 1 to 30, wherein the subject has been exposed to a source infected with SARS-CoV-2.

32. The method of claim any one of claims 1 to 31, further comprising administering to the subject an antiviral medication.

33. The method of claim 32, wherein the antiviral medication is ribavirin, neuraminidase inhibitor, protease inhibitor, recombinant interferons, antibodies, oseltamivir, zanamivir, peramivir or baloxavir marboxil.

34. The method of any one of claims 1 to 33, further comprising administering to the subject an anti-inflammatory agent.

35. The method of claim 34, wherein the anti-inflammatory agent is an NSAID or an anti-inflammatory steroid.

36. The method of claim any one of claims 1 to 35, further comprising administering to the subject hydroxychloroquine, chloroquine, remdesivir, tocilizumab and/or sarilumab.

37. A method of identifying a subject as being at risk for increased severity of a disease or condition, the method comprising determining expression levels IL-8, IL-6, IL-1β, and/or TNFα in a sample from the subject, wherein elevated expression levels of IL-8, IL-6, IL-1β, and/or TNFα in the sample indicate that the subject is at of increased severity of the disease or condition.

38. The method of claim 37, wherein the disease or condition is a coronavirus infection, an influenza virus infection, and/or a respiratory syncytial virus infection.

39. The method of claim 37, wherein the disease or condition is SARS-CoV-2 infection.

40. The method of any one of claims 37 to 39, further comprising treating the subject for the disease or condition.

41. The method of claim 40, wherein the treatment comprises orally administering to the subject a Prevotella histicola strain comprising at least 99% genomic, 16S and/or CRISPR sequence identity to the nucleotide sequence of the Prevotella Strain B 50329 (NRRL accession number B 50329).

42. The method of claim 41, wherein the Prevotella histicola strain is the Prevotella Strain B (NRRL accession number B 50329).

43. The method of any one of claims 40 to 42, wherein the treatment comprises administering the subject an antiviral medication.

44. The method of claim 43, wherein the antiviral medication is ribavirin, neuraminidase inhibitor, protease inhibitor, recombinant interferons, antibodies, oseltamivir, zanamivir, peramivir or baloxavir marboxil.

45. The method of any one of claims 40 to 44, wherein the treatment comprises administering the subject an anti-inflammatory agent.

46. The method of claim 45, wherein the anti-inflammatory agent is an NSAID or an anti-inflammatory steroid.
